(19) United States
(12) Patent Application Publication
(10) Pub. No.: US 2026/0327334 A1
(43) Pub. Date: Oct. 22, 2026

(54) DOSING REGIMEN

(71) Applicant: HaemaLogiX Ltd., Sydney NSW (AU)

(72) Inventors: Rosanne DUNN, Sydney NSW (AU); Mohamad HUSSEIN, Sydney NSW (AU); Andrew SPENCER, Sydney NSW (AU)

(21) Appl. No.: 19/355,118

(22) Filed: Oct. 10, 2025

Related U.S. Application Data
(60) Provisional application No. 63/705,884, filed on Oct. 10, 2024.

(51) Int. Cl. C07K 16/30; A61K 31/496; A61K 31/5377; A61K 31/573; A61K 39/00; A61P 35/00

(52) U.S. Cl. CPC C07K 16/3061; A61K 31/496; A61K 31/5377; A61K 31/573; A61P 35/00; A61K 2039/505; A61K 2039/545; C07K 2317/565

(57) ABSTRACT

The present disclosure relates to combination dosing regimen comprising an anti-KMA antibody, an IMiD/CELMOD and a steroid. Such combinations may be used for the treatment of multiple myeloma.

### CROSS REFERENCE TO RELATED APPLICATION

This application claims priority from U.S. Provisional Application No. 63/705,884, filed Oct. 10, 2024, the disclosure of which is incorporated herein by reference in its entirety.

### REFERENCE TO AN ELECTRONIC SEQUENCE LISTING

The contents of the electronic sequence listing (HMLX_006_01US_SeqList_ST26.xml; Size: 13,227 bytes; and Date of Creation: Oct. 9, 2025) are herein incorporated by reference in its entirety.

### FIELD OF THE INVENTION

The present disclosure relates to combination dosing regimen comprising an anti-KMA antibody, an IMiD/CELMOD and a steroid. Such combinations may be used for the treatment of multiple myeloma.

### BACKGROUND OF THE INVENTION

Multiple myeloma represents a malignant proliferation of plasma cells derived from a single clone. The multiple myeloma tumor, its products, and the patient's response to it can result in a number of organ dysfunctions, symptoms of bone pain or fracture, renal failure, susceptibility to infection, anaemia, hypocalcaemia, clotting abnormalities, neurologic symptoms and vascular manifestations of hyper viscosity.

Despite numerous treatment options, multiple myeloma remains an incurable disease for most patients with no effective long-term treatment that currently exists for multiple myeloma. For example, the current chance of surviving for 5 years post initial diagnosis in Australia and the USA is 55%. Accordingly, new methods of treatment are needed.

### SUMMARY OF THE INVENTION

Monoclonal antibodies are considered to have a high potential for immunotoxicity, which can be dose dependent. Thus, in cases where a low dose of a monoclonal antibody would be considered to be effective for therapeutic treatment, higher doses are often not contemplated.

Early investigational studies of anti-KMA antibodies suggested that they were not subject to the phenomenon known as “antigen sink”. As such, taken together with the known potential for monoclonal antibody induced immunotoxicity at higher doses, anti-KMA antibodies were considered good candidates for low dosing.

However, phase II studies surprisingly demonstrated that administration of an anti-KMA antibody at a dose of 10 mg/kg provided an advantageous safety profile in human subjects. This advantageous safety profile was a result of the anti-KMA antibody unexpectedly not depleting immune cells, either alone or, more particularly in conjunction with an immunomodulatory drug (IMiD) or cereblon E3 ligase modulator (CELMOD) and a steroid. In addition, when kappaMab (KM) was administered in conjunction with an IMiD and a steroid (e.g., lenalidomide/dexamethasone), the safety profile of this combination was comparable to that of lenalidomide/dexamethasone administered in combination in the literature (e.g., there were no increases in rates of thrombocytopenia, neutropenia or infection in the KM/lenalidomide/dexamethasone treated patients compared to lenalidomide/dexamethasone alone).

The observed safety profile is surprising because, administration of other monoclonal antibodies in the treatment of multiple myeloma typically results in depletion of immune cells due to the antigen target being present on both the malignant plasma cells and on healthy immune cells (e.g., BCMA and CD38). Thus, the present inventor's findings support higher dosing of anti-KMA antibodies (e.g., above 10 mg/kg), such as KM, and particularly in conjunction with an IMiD or a CELMOD and a steroid. This is a significant advance in the art because increased anti-KMA antibody dosing, in particular in conjunction with and an IMiD or a CELMOD and a steroid may improve outcomes in patients with multiple myeloma, in particular those patients whose multiple myeloma has relapsed or is refractory.

Accordingly, in one example, the present disclosure relates to a method of treating multiple myeloma in a subject in need thereof, the method comprising administering to the subject a treatment which comprises:
at least 30 mg/kg of an anti-KMA antibody;
an IMiD or a CELMOD; and
a steroid.

In another example, the present disclosure relates to a method of adjuvant therapy comprising administering to a subject having multiple myeloma, following or in combination with primary treatment, an effective amount of an anti-KMA antibody. In some cases, the effective amount is about 30 mg/kg to about 60 mg/kg of the anti-KMA antibody. In some cases, the effective amount is 30 mg/kg to 60 mg/kg of the anti-KMA antibody. In some cases, the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody. In some cases, the effective amount is at least 10 mg/kg of the anti-KMA antibody. In some cases, the effective amount is at least 30 mg/kg of the anti-KMA antibody. In some cases, the effective amount is at least 45 mg/kg of the anti-KMA antibody. In some cases, the effective amount is at least 60 mg/kg of the anti-KMA antibody. In some cases, the primary treatment is at least one proteasome inhibitor, IMiD, CELMOD or autologous stem cell transplantation. In some cases, the primary treatment is any line of therapy known in the art for treating multiple myeloma. In some cases, the primary treatment comprises daratumumab or daratumumab in combination with hyaluronidase. In some cases, the primary treatment comprises a combination of an IMiD or a CELMOD and a steroid. In some cases, the IMiD is lenalidomide, pomalidomide or thalidomide. In some cases, the IMiD can be lenalidomide. In some cases, the IMiD can be pomalidomide. In some cases, the steroid is dexamethasone or prednisone. In some cases, the steroid is dexamethasone. In some cases, the primary treatment comprises a combination of pomalidomide and dexamethasone. In some cases, the IMiD or CELMOD is administered at a dose provided herein. In some cases, the IMiD or CELMOD is administered according to a dosing regimen provided herein. In some cases, the steroid is administered at a dose provided herein. In some cases, the steroid is administered according to a dosing regimen provided herein. In some cases, the daratumumab or daratumumab in combination with hyaluronidase is administered at a dose provided herein. In some cases, the daratumumab or daratumumab in combination with hyaluronidase is administered according to a dosing regimen provided herein. In some cases, the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks. In some cases, the anti-KMA antibody is administered once per month for at least 1 month, at least 2 months, at least 3 months, at least 4 months, at least 5 months or at least 6 months. For example, the duration of the adjuvant therapy is 12 weeks. In some cases, the adjuvant therapy continues until disease progression.

In another example, the present disclosure relates to an adjuvant method of treating multiple myeloma in a subject, the method comprising administering an effective amount of an anti-KMA antibody in combination with an existing therapeutic protocol. In some cases, the effective amount is about 30 mg/kg to about 60 mg/kg of the anti-KMA antibody. In some cases, the effective amount is 30 mg/kg to 60 mg/kg of the anti-KMA antibody. In some cases, the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody. In some cases, the effective amount is at least 10 mg/kg of the anti-KMA antibody. In some cases, the effective amount is at least 30 mg/kg of the anti-KMA antibody. In some cases, the effective amount is at least 45 mg/kg of the anti-KMA antibody. In some cases, the effective amount is at least 60 mg/kg of the anti-KMA antibody. In some cases, the existing therapeutic protocol comprises administering at least one proteasome inhibitor, IMiD, CELMOD, daratumumab, daratumumab in combination with hyaluronidase or autologous stem cell transplantation. In some cases, the existing therapeutic protocol comprises administering a combination of an IMiD or a CELMOD and a steroid. In some cases, the existing therapeutic protocol comprises administering daratumumab as a monotherapy or daratumumab as a combination therapy with an IMiD, a proteasome inhibitor, an alkylating agent, a steroid or any combination thereof. In some cases, the IMiD is lenalidomide, pomalidomide or thalidomide. In some cases, the IMiD can be lenalidomide. In some cases, the IMiD can be pomalidomide. In some cases, the steroid is dexamethasone or prednisone. In some cases, the steroid is dexamethasone. In some cases, the alkylating agent is melphalan. In some cases, the proteasome inhibitor is bortezomib. In some cases, the proteasome inhibitor is carfilzomib. In some cases, the existing therapeutic protocol comprises administering a combination of pomalidomide and dexamethasone. In some cases, the existing therapeutic protocol comprises administering a combination of daratumumab, pomalidomide and dexamethasone. In some cases, the existing therapeutic protocol comprises administering a combination of daratumumab, lenalidomide and dexamethasone. In some cases, the existing therapeutic protocol comprises administering a combination of daratumumab, bortezomib, melphalan and prednisone. In some cases, the existing therapeutic protocol comprises administering a combination of daratumumab, bortezomib, thalidomide, and dexamethasone. In some cases, the existing therapeutic protocol comprises administering a combination of daratumumab, bortezomib and dexamethasone. In some cases, the existing therapeutic protocol comprises administering a combination of daratumumab, carfilzomib and dexamethasone. In some cases, the IMiD or CELMOD is administered at a dose provided herein. In some cases, the IMiD or CELMOD is administered according to a dosing regimen provided herein. In some cases, the steroid is administered at a dose provided herein. In some cases, the steroid is administered according to a dosing regimen provided herein. In some cases, the daratumumab or daratumumab in combination with hyaluronidase is administered at a dose provided herein. In some cases, the daratumumab or daratumumab in combination with hyaluronidase is administered according to a dosing regimen provided herein. In some cases, the alkylating agent is administered at a dose provided herein. In some cases, the alkylating agent is administered according to a dosing regimen provided herein. In some cases, the proteasome inhibitor is administered at a dose provided herein. In some cases, the proteasome inhibitor is administered according to a dosing regimen provided herein. In some cases, the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks. In some cases, the anti-KMA antibody is administered once per month for at least 1 month, at least 2 months, at least 3 months, at least 4 months, at least 5 months or at least 6 months. For example, the duration of the adjuvant therapy is 12 weeks. In some cases, the adjuvant therapy continues until disease progression. In some cases, the multiple myeloma (MM) is a relapsed or refractory MM. In some cases, the multiple myeloma is relapsed refractory multiple myeloma (RRMM).

The methods of the present disclosure also relate to a combination dosing regimen for use in the treatment of multiple myeloma in a subject comprising:
at least 30 mg/kg of an anti-KMA antibody;
an IMiD or a CELMOD; and
a steroid.

The methods of the present disclosure further relate to a kit comprising:
a single dose or multiple doses of at least 30 mg/kg of an anti-KMA antibody;
a single dose or multiple doses of an IMiD or a CELMOD;
a single dose or multiple doses of a steroid;
instructions for using the anti-KMA antibody, the IMiD or the CELMOD, and the steroid, for the treatment of multiple myeloma in a subject.

The methods of the present disclosure also relate to a combination dosing regimen for use in the treatment of multiple myeloma in a subject comprising:
at least 8 mg/kg or at least 16 mg/kg of daratumumab or at least 1800 mg daratumumab/30,000 units hyaluronidase;
at least 30 mg/kg of an anti-KMA antibody;
an IMiD or a CELMOD; and
a steroid.

The methods of the present disclosure further relate to a kit comprising:
a single dose or multiple doses of at least 8 mg/kg or at least 16 mg/kg of daratumumab or at least 1800 mg daratumumab/30,000 units hyaluronidase;
a single dose or multiple doses of at least 30 mg/kg of an anti-KMA antibody;
a single dose or multiple doses of an IMiD or a CELMOD;
a single dose or multiple doses of a steroid;
instructions for using the anti-KMA antibody, the IMiD or the CELMOD, and the steroid, for the treatment of multiple myeloma in a subject.

In the above examples, the anti-KMA antibody comprises a heavy chain variable region (VH) and a light chain variable region (VL), the VH comprising a complementarity determining region (CDR) 1 comprising an amino acid sequence as shown in SEQ ID NO: 6, a CDR2 comprising an amino acid sequence as shown in SEQ ID NO: 7 and a CDR3 comprising a sequence as shown in SEQ ID NO: 8 and the VL comprising a CDR 1 comprising an amino acid sequence as shown in SEQ ID NO: 9, a CDR2 comprising an amino acid sequence as shown in SEQ ID NO: 10 and a CDR3 comprising a sequence as shown in SEQ ID NO: 11. In another example, the anti-KMA antibody has a VH comprising a sequence set forth in SEQ ID NO: 1 and a VL comprising a sequence set forth in SEQ ID NO: 2.

In an example, about 30 mg/kg to about 60 mg/kg of the anti-KMA antibody is administered to the subject. For example, 30 mg/kg to 60 mg/kg of the anti-KMA antibody is administered to the subject. In an example, about 30 mg/kg of the anti-KMA antibody is administered to the subject. For example, 30 mg/kg of the anti-KMA antibody is administered to the subject. In another example, about 45 mg/kg of the anti-KMA antibody is administered to the subject. For example, 45 mg/kg of the anti-KMA antibody is administered to the subject. In a further example, about 60 mg/kg of the anti-KMA antibody is administered to the subject. For example, 60 mg/kg of the anti-KMA antibody is administered to the subject.

In an example, the IMiD is lenalidomide, pomalidomide or thalidomide. For example, the IMiD can be lenalidomide. In an example, the CELMOD is iberdomide or CC-92480. For example, the CELMOD is iberdomide. In an example, about 1 mg to 25 mg of the IMiD or the CELMOD is administered to the subject. In an example, about 25 mg of the IMiD or the CELMOD is administered to the subject. In an example, 25 mg of the IMiD or the CELMOD is administered to the subject. For example, about 25 mg of lenalidomide is administered to the subject. In one example, 25 mg of lenalidomide is administered to the subject. In one example, 4 mg of pomalidomide is administered to the subject. In one example, 3 mg of pomalidomide is administered to the subject. In one example, 2 mg of pomalidomide is administered to the subject. In one example, 1 mg of pomalidomide is administered to the subject.

In an example, the steroid is dexamethasone or prednisone. For example, the steroid is dexamethasone. In an example, about 40 mg of the steroid is administered to the subject. In an example, 40 mg of the steroid is administered to the subject. For example, about 40 mg of dexamethasone is administered to the subject. In one example, 40 mg of dexamethasone is administered to the subject. In one example, 20 mg of dexamethasone is administered to the subject. In one example, 10 mg of dexamethasone is administered to the subject.

In an example a method of treating multiple myeloma in a subject in need thereof, the method comprising administering to the subject a treatment which comprises:
at least 30 mg/kg of an anti-KMA antibody;
25 mg of an IMiD or a CELMOD; and
40 mg of steroid.

In an example a method of treating multiple myeloma in a subject in need thereof, the method comprising administering to the subject a treatment which comprises:
at least 30 mg/kg of an anti-KMA antibody having the CDRs of KM;
25 mg of lenalidomide; and
40 mg of dexamethasone.

In an example a method of treating multiple myeloma in a subject in need thereof, the method comprising administering to the subject a treatment which comprises:
at least 30 mg/kg of an anti-KMA antibody having the CDRs of KM;
between 1 mg to 4 mg of pomalidomide; and
between 10 mg to 40 mg of dexamethasone.

In an example a method of treating multiple myeloma in a subject in need thereof, the method comprising administering to the subject a treatment which comprises:
at least 30 mg/kg of an anti-KMA antibody having the CDRs of KM;
4 mg of pomalidomide; and
40 mg of dexamethasone.

In an example a method of treating multiple myeloma in a subject in need thereof, the method comprising administering to the subject a treatment which comprises:
at least 30 mg/kg of an anti-KMA antibody having the CDRs of KM;
3 mg of pomalidomide; and
20 mg of dexamethasone.

In an example, treatment comprises administering to the subject in a treatment phase:
at least 30 mg/kg of the anti-KMA antibody once per week;
the IMiD or the CELMOD once per day; and
the steroid once per week.

In an example a method of treating multiple myeloma in a subject in need thereof, the method comprising administering to the subject a treatment which comprises:
at least 8 mg/kg or at least 16 mg/kg of daratumumab or at least 1800 mg daratumumab/30,000 units hyaluronidase;
at least 30 mg/kg of an anti-KMA antibody;
25 mg of an IMiD or a CELMOD; and
40 mg of steroid.

In an example a method of treating multiple myeloma in a subject in need thereof, the method comprising administering to the subject a treatment which comprises:
at least 8 mg/kg or at least 16 mg/kg of daratumumab or at least 1800 mg daratumumab/30,000 units hyaluronidase;
at least 30 mg/kg of an anti-KMA antibody having the CDRs of KM;
25 mg of lenalidomide; and
40 mg of dexamethasone.

In an example a method of treating multiple myeloma in a subject in need thereof, the method comprising administering to the subject a treatment which comprises:
at least 8 mg/kg or at least 16 mg/kg of daratumumab or at least 1800 mg daratumumab/30,000 units hyaluronidase;
at least 30 mg/kg of an anti-KMA antibody having the CDRs of KM;
between 1 mg to 4 mg of pomalidomide; and
between 10 mg to 40 mg of dexamethasone.

In an example a method of treating multiple myeloma in a subject in need thereof, the method comprising administering to the subject a treatment which comprises:
at least 8 mg/kg or at least 16 mg/kg of daratumumab or or at least 1800 mg daratumumab/30,000 units hyaluronidase;
at least 30 mg/kg of an anti-KMA antibody having the CDRs of KM;
4 mg of pomalidomide; and
40 mg of dexamethasone.

In an example a method of treating multiple myeloma in a subject in need thereof, the method comprising administering to the subject a treatment which comprises:
at least 8 mg/kg or at least 16 mg/kg of daratumumab or at least 1800 mg daratumumab/30,000 units hyaluronidase;
at least 30 mg/kg of an anti-KMA antibody having the CDRs of KM;
3 mg of pomalidomide; and 20 mg of dexamethasone.

In an example, treatment comprises administering to the subject in a treatment phase: at least 8 mg/kg or at least 16 mg/kg of daratumumab once per week or at least 1800 mg daratumumab/30,000 units hyaluronidase per week;
at least 30 mg/kg of the anti-KMA antibody once per week;
the IMiD or the CELMOD once per day; and
the steroid once per week.

In an example, the duration of the treatment phase is between at least 6 weeks and at least 10 weeks. For example, the duration of the treatment phase is between 6 weeks and 10 weeks. In an example, the duration of the treatment phase is at least 8 weeks. For example, the duration of the treatment phase is 8 weeks.

In an example, the methods of the present disclosure further comprise a neoadjuvant phase that comprises administering to the subject an effective amount of the anti-KMA antibody prior to a treatment phase as provided herein or an induction phase as provided herein. In some cases, the effective amount is about 30 mg/kg to about 60 mg/kg of the anti-KMA antibody. In some cases, the effective amount is 30 mg/kg to 60 mg/kg of the anti-KMA antibody. In some cases, the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody. In some cases, the effective amount is at least 10 mg/kg of the anti-KMA antibody. In some cases, the effective amount is at least 30 mg/kg of the anti-KMA antibody. In some cases, the effective amount is at least 45 mg/kg of the anti-KMA antibody. In some cases, the effective amount is at least 60 mg/kg of the anti-KMA antibody. In some cases, the induction phase comprises administering a composition dosing regimen provided herein. In some cases, the induction phase comprises administering a composition dosing regimen that comprises an IMiD or a CELMOD and a steroid. In some cases, the treatment phase comprises administering a composition dosing regimen that comprises an IMiD or a CELMOD and a steroid. In some cases, the IMiD is lenalidomide, pomalidomide or thalidomide. In some cases, the IMiD can be lenalidomide. In some cases, the IMiD can be pomalidomide. In some cases, the steroid is dexamethasone or prednisone. In some cases, the steroid is dexamethasone. In some cases, the IMiD or CELMOD is administered at a dose provided herein. In some cases, the steroid is administered at a dose provided herein. In some cases, the IMiD or CELMOD is administered according to a dosing regimen provided herein. In some cases, the steroid is administered according to a dosing regimen provided herein. In some cases, the neoadjuvant phase comprises administering the anti-KMA antibody once per week for at least 8 weeks prior to the start of the induction phase. In some cases, the neoadjuvant phase comprises administering the anti-KMA antibody once per week for at least 8 weeks prior to the start of the treatment phase.

In an example, the methods of the present disclosure further comprise administering to the subject the IMiD or the CELMOD once per day for at least 3 weeks prior to the start of the treatment phase. For example, the methods of the present disclosure further comprise administering to the subject the IMiD or the CELMOD once per day for 3 weeks prior to the start of the treatment phase.

In an example, the methods of the present disclosure further comprise administering a steroid to the subject at least 1 week prior to the start of the treatment phase. For example, the methods of the present disclosure further comprise administering a steroid to the subject 1 week prior to the start of the treatment phase.

In an example, the subject's immune cells are maintained at levels corresponding with their immune cells prior to administration of the anti-KMA antibody. In another example, the subject's immune cells are maintained at levels corresponding with their immune cells after the treatment phase. In an example, the subject's immune cells are one or more or all of red blood cells; white blood cells; and platelets.

In an example, treatment comprises administering to the subject in a treatment phase:
at least 30 mg/kg of the anti-KMA antibody once per week;
25 mg of the IMiD or the CELMOD once per day; and
40 mg of the steroid once per week.

In an example, treatment comprises administering to the subject in a treatment phase:
at least 30 mg/kg of the anti-KMA antibody once per week;
between 1 mg to 5 mg of the IMiD or the CELMOD once per day; and
between 10 mg to 40 mg of the steroid once per week.

In an example, treatment comprises administering to the subject in a treatment phase:
at least 30 mg/kg of an anti-KMA antibody having the CDRs of KM once per week;
25 mg of lenalidomide once per day; and
40 mg of dexamethasone once per week.

In an example, treatment comprises administering to the subject in a treatment phase:
at least 30 mg/kg of an anti-KMA antibody having the CDRs of KM once per week;
1 mg to 4 mg of pomalidomide once per day; and
10 mg to 40 mg of dexamethasone once per week.

In an example, treatment comprises administering to the subject in a treatment phase:
at least 30 mg/kg of an anti-KMA antibody having the CDRs of KM once per week;
4 mg of pomalidomide once per day; and
40 mg of dexamethasone once per week.

In an example, treatment comprises administering to the subject in a treatment phase:
at least 30 mg/kg of an anti-KMA antibody having the CDRs of KM once per week;
2 mg of pomalidomide once per day; and
20 mg of dexamethasone once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of the anti-KMA antibody once per week;
25 mg of the IMiD or the CELMOD once per day; and
40 mg of the steroid once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of the anti-KMA antibody once per week;
between 1 mg to 5 mg of the IMiD or the CELMOD once per day; and
between 10 mg to 40 mg of the steroid once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of an anti-KMA antibody having the CDRs of KM once per week;
25 mg of lenalidomide once per day; and
41 mg of dexamethasone once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of an anti-KMA antibody having the CDRs of KM once per week;
1 mg to 4 mg of pomalidomide once per day; and
10 mg to 40 mg of dexamethasone once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of an anti-KMA antibody having the CDRs of KM once per week;
4 mg of pomalidomide once per day; and
40 mg of dexamethasone once per week.

In an example, treatment comprises administering to the subject in a treatment phase:
at least 8 mg/kg or at least 16 mg/kg of daratumumab or at least 1800 mg daratumumab/30,000 units hyaluronidase once per week;
at least 30 mg/kg of an anti-KMA antibody having the CDRs of KM once per week;
2 mg of pomalidomide once per day; and
20 mg of dexamethasone once per week.

In an example, the methods of the present disclosure further comprise an adjuvant or adjunct phase which comprises administering to the subject an effective amount of the anti-KMA antibody once per week or an effective amount of the anti-KMA antibody once per month. In some cases, the anti-KMA antibody has the CDRs of KM. In some cases, the effective amount is about 30 mg/kg to about 60 mg/kg of the anti-KMA antibody. In some cases, the effective amount is 30 mg/kg to 60 mg/kg of the anti-KMA antibody. In some cases, the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody. In some cases, the effective amount is at least 10 mg/kg of the anti-KMA antibody. In some cases, the effective amount is at least 30 mg/kg of the anti-KMA antibody. In some cases, the effective amount is at least 45 mg/kg of the anti-KMA antibody. In some cases, the effective amount is at least 60 mg/kg of the anti-KMA antibody.

In an example, the adjuvant phase follows after the treatment phase. In an example, the duration of the adjuvant phase is at least 4 weeks, at least 8 weeks, at least 12 weeks, at least 16 weeks, at least 20 weeks, at least 24 weeks, at least 28 weeks, at least 32 weeks or at least 36 weeks, when the anti-KMA antibody is administered once per week. For example, the duration of the adjuvant therapy is 12 weeks. In some cases, the duration of the adjuvant phase is at least 1 month, at least 2 months, at least 3 months, at least 4 months, at least 5 months or at least 6 months, when the anti-KMA antibody is administered once per month. In some cases, the adjuvant phase continues until disease progression.

In an example, the methods of the present disclosure further comprise a maintenance phase which comprises administering to the subject:
at least 30 mg/kg of the anti-KMA antibody once per month;
the IMiD or the CELMOD once per day; and
the steroid once per week.

In an example, the methods of the present disclosure further comprise a maintenance phase which comprises administering to the subject:
at least 30 mg/kg of the anti-KMA antibody once per month;
25 mg of the IMiD or the CELMOD once per day; and
40 mg of the steroid once per week.

In an example, the methods of the present disclosure further comprise a maintenance phase which comprises administering to the subject:
at least 30 mg/kg of the anti-KMA antibody once per month;
between 1 mg to 4 mg of the IMiD or the CELMOD once per day; and
between 10 mg to 40 mg of the steroid once per week.

In an example, the methods of the present disclosure further comprise a maintenance phase which comprises administering to the subject:
at least 30 mg/kg of the anti-KMA antibody once per month;
4 mg of the IMiD or the CELMOD once per day; and
40 mg of the steroid once per week.

In an example, the methods of the present disclosure further comprise a maintenance phase which comprises administering to the subject:
at least 30 mg/kg of the anti-KMA antibody once per month;
3 mg of the IMiD or the CELMOD once per day; and
20 mg of the steroid once per week.

In an example, the methods of the present disclosure further comprise a maintenance phase which comprises administering to the subject:
at least 30 mg/kg of an anti-KMA antibody having the CDRs of KM once per week;
25 mg of lenalidomide once per day; and
40 mg of dexamethasone once per week.

In an example, the methods of the present disclosure further comprise a maintenance phase which comprises administering to the subject:
at least 30 mg/kg of an anti-KMA antibody having the CDRs of KM once per week;
4 mg of pomalidomide once per day; and
40 mg of dexamethasone once per week

In an example, the methods of the present disclosure further comprise a maintenance phase which comprises administering to the subject:
at least 30 mg/kg of an anti-KMA antibody having the CDRs of KM once per week;
3 mg of pomalidomide once per day; and
20 g of dexamethasone once per week

In an example, the maintenance phase follows after the treatment phase. In an example, the duration of the maintenance phase is at least 12 weeks. For example, the duration of the maintenance phase is 12 weeks. In another example, the maintenance phase continues until disease progression.

After a subject reaches disease progression, the subject is taken off the therapy. In one example, the subject is put onto another anti-multiple myeloma therapy. In another example, the maintenance phase is stopped.

In an example, the anti-KMA antibody is formulated for intravenous infusion. In an example, the IMiD or the CELMOD is formulated for oral delivery. In an example, the steroid is formulated for oral delivery or intravenous infusion.

In an example, the subject has received at least one, at least two, at least three, at least four, at least five or at least six prior lines of therapy.

In an example, the subject achieved at least a minimal response (e.g., a 25% reduction in M protein) to their most recent line of therapy.

In an example, the subject is refractory to at least one, at least two, at least three or at least four prior lines of therapy. In an example, the subject is refractory to at least one proteasome inhibitor, IMID, CELMOD, monoclonal antibody and/or autologous stem cell transplantation. In an example, the subject is refractory to at least two prior lines of therapy. In an example, the subject is refractory to bortezomib. In an example, the subject is refractory to daratumumab.

In an example, the subject has relapsed myeloma. In some cases, the multiple myeloma has relapsed or is refractory. In some cases, the multiple myeloma is relapsed refractory multiple myeloma (RRMM). In an example, the subject's multiple myeloma has relapsed and is refractory to at least one proteasome inhibitor, IMiD, CELMOD, monoclonal antibody and/or autologous stem cell transplantation.

In an example, the subject has no prior exposure to the IMiD or the CELMOD. For example, the subject has no prior exposure to lenalidomide.

Any example herein shall be taken to apply mutatis mutandis to any other example unless specifically stated otherwise.

The present invention is not to be limited in scope by the specific examples described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying drawings.

### KEY TO SEQUENCE LISTING

SEQ ID NO: 1-amino acid sequence of KappaMab variable heavy chain (VH)
SEQ ID NO: 2-amino acid sequence of KappaMab variable light chain (VL)
SEQ ID NO: 3-amino acid sequence of KMA switch region in kappa light chain
SEQ ID NO: 4-amino acid sequence of KappaMab epitope
SEQ ID NO: 5-amino acid sequence of KappaMab epitope 2 (improved binding)
SEQ ID NO: 6-amino acid sequence of KappaMab VH CDR1
SEQ ID NO: 7-amino acid sequence of KappaMab VH CDR2
SEQ ID NO: 8-amino acid sequence of KappaMab VH CDR3
SEQ ID NO: 9-amino acid sequence of KappaMab VL CDR1
SEQ ID NO: 10-amino acid sequence of KappaMab VL CDR2
SEQ ID NO: 11-amino acid sequence of KappaMab VL CDR3
SEQ ID NO: 12-amino acid sequence of K121 VH
SEQ ID NO: 13-amino acid sequence of K121 VC

### BRIEF DESCRIPTION OF THE ACCOMPANYING DRAWINGS

FIG. 1 shows the study schema. Stage 1 involved 8 weekly doses of 10 mg/kg KappaMab, followed by monthly dosing until progression. Stage 2 was 8 weekly doses of 10 mg/kg KappaMab plus 25 mg lenalidomide and 40 mg dexamethasone, followed by monthly dosing of KappaMab plus lenalidomide and dexamethasone until progression. In Stage 2, Cycle 1 was 35 days, with a one-week administration of LEN and DEX prior to the first dose of KappaMab. The remaining cycles were 28 days, with KappaMab administration on Day 1 of each Cycle.

FIG. 2 shows the duration of response in Stage KM-Rd treated patients. Shaded region indicates the 95% confidence interval for the probability of remaining in response at each time point.

FIG. 3 shows the cumulative incidences for the occurrence of the next treatment or the competing event of death before next treatment. Shaded regions indicate the 95% confidence intervals for each type of event at each time point.

FIG. 4 shows ORR and best clinical response rate comparisons between Stage 2 KM-Rd and the Rd-MRDR patients. A comparison of the best clinical responses for the Rd-MRDR group with patients in Stage 2 showed that Stage 2 patients overall had higher percentages of best clinical responses with more responses and improved quality of response. In addition, Stage 2 (KM-Rd patients) had an ORR of 83% and a CBR of 93% compared to an ORR of 45% in matched controls from the Australian and New Zealand Myeloma and Related Diseases registry (Rd-MRDR group).

FIG. 5 shows comparisons of the time-to-event overall survival endpoint comparing the KM-Rd group with Rd-MRDR group. The logrank test showed a significance of p=0.0163, and the hazard ratio with 95% confidence interval was HR 0.46, 95% CI (0.2466, 0.8679). Median OS in the Rd-MRDR group was 27.8 months, and median OS was not reached in the KM-Rd group. For mortality data, in the KM-Rd cohort (n=40), 9 patients had evaluable data and 31 patients were censored, while in the Rd-MRDR group (n=77), 32 patients had evaluable data and 45 patients were censored.

FIGS. 6A-D shows serial M protein and FLC levels for Stage 1 (FIGS. 6A and 6B) and Stage 2 (FIGS. 6C and 6D). In Stage 1, M protein levels decreased below baseline for 1 patient (FIG. 6A, open upside down triangles), but FLC increased for this patient (FIG. 6B). In Stage 1, one FLC-only MM patient showed decreased sFLC levels ranging between −36% and −80% from baseline for 31 cycles of treatment (FIG. 6B, open dotted circles). In Stage 2, the majority of patients responded to KM-Rd (FIG. 6C). Median % change from baseline for FLC was elevated to Cycle 3 (i.e., after 9 doses of KM), then it reduced and remained below baseline (FIG. 6D red line). This was not observed in the Stage 1 patients, where median FLC fell between Cycles 3 and 4, then generally elevated followed Cycle 4 (FIG. 6B red line). Median is a white line that is marked by an arrow in the Figure.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, oncology and clinical studies).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

One of skill in the art will be aware that an “antibody” is generally considered to be a protein that comprises a variable region made up of a plurality of polypeptide chains, e.g., a polypeptide comprising a VL and a polypeptide comprising a VH. An antibody also generally comprises constant domains, some of which can be arranged into a constant region or constant fragment or fragment crystallizable (Fc). A VH and a VL interact to form a Fv comprising an antigen binding region that specifically binds to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgAQ1 and IgA2) or subclass. The term “antibody” also encompasses humanized antibodies, primatized antibodies, human antibodies and chimeric antibodies.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

An “antigen binding fragment” of an antibody comprises one or more variable regions of an intact antibody. Examples of antibody fragments include Fab, Fab′, F(ab′) 2 and Fv fragments (scFv, di-scFv, tri-scFv); diabodies; linear antibodies; single-chain antibody molecules and multispecific antibodies formed from antibody fragments.

The term “complementarity determining regions” (syn. CDRs; i.e., CDR1, CDR2, and CDR3) is used in the context of the present disclosure to refer to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27:55-77), Chothia (Chothia C, Lesk A M (1987), J Mal Biol 196:901-917; Chothia, et al. (1989), Nature 342:877-883) and AHo (Honegger A, Plückthun A (2001) J Mol Biol 309:657-670). For convenience, examples of binding proteins of the present disclosure may also be labelled according to IMGT.

The term “antibody heavy chain” is used herein to refer to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

As used herein, the term “binds” in reference to the interaction of an antibody or antigen binding fragment thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the antibody, will reduce the amount of labelled “A” bound to the antibody.

As used herein, the term “specifically binds” shall be taken to mean that the binding interaction between an antibody or antigen binding fragment thereof and kappa myeloma antigen is dependent on the presence of the antigenic determinant or epitope of kappa myeloma antigen bound by the antibody or antigen binding fragment thereof. Accordingly, the antibody or antigen binding fragment thereof preferentially binds or recognizes a kappa myeloma antigen determinant or epitope even when present in a mixture of other molecules or organisms. In one example, the antibody or antigen binding fragment thereof reacts or associates more frequently, more rapidly, with greater duration and/or with greater affinity with kappa myeloma antigen or a cell expressing the same than it does with alternative antigens or cells. It is also understood by reading this definition that, for example, an antibody or antigen binding fragment thereof that specifically binds to kappa myeloma antigen may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term.

Methods for determining specific binding will be apparent to the skilled person. In an example, an anti-KMA antibody according to the present disclosure is contacted with kappa myeloma antigen or a cell expressing same or a mutant form thereof or an alternative antigen. The binding of the antibody to the kappa myeloma antigen or mutant form or alternative antigen is then determined and an antibody that binds as set out above to the kappa myeloma antigen rather than the mutant or alternative antigen is considered to specifically bind to kappa myeloma antigen.

The term “antigen sink” is used in the context of the present disclosure to describe a monoclonal antibody that binds to an unintended target (e.g., healthy cells) that prevents the antibodies from reaching the intended tumor antigen targets in vivo as these unintended targets will “sop up” the antibody, serving as a sink

The phrase “a combination dosing regimen” is used in the context of the present disclosure to refer to a combination of therapeutics that are administered to a subject according to a dosing regimen disclosed herein.

As used herein, the term “subject” or “patient” may be used interchangeably and shall be taken to mean any animal including humans, for example, a mammal. Exemplary subjects include but are not limited to humans and non-human primates. For example, the subject is a human.

The phrase “therapeutic” is used in the context of the present disclosure to refer to a treatment, therapy, or drug.

One of skill in the art would appreciate that the therapeutics disclosed herein are generally administered in phases. For example, a “treatment phase” refers to a period of time that is used to gain rapid control the multiple myeloma and relieve symptoms. In certain examples, there is an induction phase prior to the treatment phase.

The phrase “induction phase” as used herein is used to refer to a period of time before the treatment phase starts.

In certain examples, the treatment phase is followed by a maintenance phase. The phrase “maintenance phase” as used herein is used to refer to a period of time in which treatment of the therapeutic is continued in order to inhibit or, in certain examples, prevent recurrence of the multiple myeloma.

As used herein, the term “adjuvant” can be used to refer to a drug or other substance such as, for example, an anti-KMA antibody e.g., KappaMab or KM, that is used to increase the efficacy or potency of other drugs and/or improve patient outcomes. For example, an anti-KMA antibody as provided herein can act as an adjuvant that enhances or increases the potency of other drugs such as, for example, a combination dosing regimen comprising an IMiD or CELMOD along with a steroid. In one example, an anti-KMA antibody as provided herein may act as an adjuvant that enhances or improves patient outcomes.

As used herein, the phrase “adjuvant therapy” can be used to refer to, in the broadest sense, treatment, such as, for example, treatment with an anti-KMA antibody e.g., KappaMab or KM, given after or in addition to the primary therapy to kill or aid in killing any malignant tumor cells that may have spread, even if the spread cannot be detected by radiologic or laboratory tests and/or to prevent recurrence of a cancer such as, for example, multiple myeloma. The primary treatment can be any line of therapy known in the art for use in treating a cancer such as, for example, multiple myeloma. The primary treatment can be an IMiD or CELMOD, a proteasome inhibitor and a steroid. For example, the primary treatment can be lenalidomide, bortezomib and dexamethasone. In one embodiment, the composition dosing regimen comprises an IMiD or CELMOD in combination with a steroid. The terms “adjuvant therapy” or “adjunct therapy” or “adjuvant care” can be used interchangeably herein.

As used herein, the phrase “neoadjuvant therapy” can be used to refer to, in the broadest sense, any treatment given prior to the primary therapy to kill or aid in killing any cancer cells and improve the chances of successful treatment of multiple myeloma. The primary treatment can be any line of therapy known in the art for use in treating multiple myeloma.

As used herein, the terms “once per day” or “daily” in the context of dosing refers to a total dose of a drug (e.g. lenalidomide or pomalidomide) administered to a subject in a day. The dose may be divided to two or more administrations during the day, or given as one administration per day. For example, the total dose may be 25 mg administered daily as a single dose. In another example, the total dose may be between 1 mg to 4 mg administered daily as a single dose.

As used herein, the terms “once per week”, “weekly” or “at least one week” in the context of dosing refers to an approximate number, and can include every seven days±two days, i.e., every five days to every nine days. The dosing frequency of “once per week” can be every five days, every six days, every seven days, every eight days, or every nine days.

As used herein, the terms “once per month”, “monthly” or “at least four weeks” in the context of dosing refers to an approximate number, and can include every 28 days±1 week, i.e., every 21 to every 35 days. The dosing frequency of “once per month” can be every 21 days, 22 days, 23 days, 24 days, 25 days, 26 days, every 27 days, every 28 days, every 29 days, every 30 days, every 31 days, every 32 days, every 33 days, every 34 days or every 35 days.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

The term “analyte” is used in the context of the present disclosure to refer to a molecule whose presence in a sample provides a quantitative or qualitative measure of gene expression. Exemplary analytes informative of gene expression levels include RNA and protein. Various methods of determining RNA and protein levels are known in the art. Exemplary methods include whole genome sequencing, next generation sequencing, NanoString technology, droplet digital PCR, quantitative RT-PCR, mass spectrometry, immunohistochemistry and multiplex immunoassay. In an example, the analyte is a cytokine which is measured using a multiplex immunoassay (e.g. Bio-Plex Pro Human Cytokine assay; Bio-Rad).

As used in this specification and the appended claims, terms in the singular and the singular forms “a,” “an” and “the,” for example, optionally include plural referents unless the content clearly dictates otherwise.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Combination Dosing Regimen

### Therapeutics

The phrase “anti-KMA antibody” is used in the context of the present disclosure to refer to an antibody that binds or specifically binds Kappa Myeloma Antigen (KMA). KMA is a membrane-bound light chain with selectivity for kappa myeloma cells (Boux, H A. et al. (1983) J Exp Med. 158:1769). KMA is a unique cell-surface antigen expressed on malignant plasma cells in kappa restricted multiple myeloma (KMM), some lymphomas, occasional tonsillar B cells and in-vitro activated B cells, but not on normal B cells in bone marrow.

In an example, an anti-KMA antibody is capable of binding KMA bearing cells. In another example, an anti-KMA antibody is capable of killing KMA bearing cells. For example, anti-KMA antibodies according to the present disclosure can bind and kill KMA bearing malignant plasma cells. In an example, anti-KMA antibodies according to the present disclosure do not bind intact immunoglobulin. Put another way, exemplary anti-KMA antibodies do not recognise kappa light chains that are in association with Ig heavy chain such as in an intact Ig molecules.

In an example, anti-KMA antibodies of the disclosure do not bind kappa light chain associated with heavy chain. For example, anti-KMA antibodies do not bind the kappa light chain of an antibody which also comprises a heavy chain.

For example, the anti-KMA antibody can be the “K121 antibody” disclosed in (Hutchinson et al. 2011) or a variant, antigen binding fragment or humanised form thereof. An exemplary humanized form is referred to in the context of the present disclosure as “kappaMab” or “KM” used interchangeably, an antibody having a heavy chain variable region (VH) comprising the amino acid sequence set forth in SEQ ID NO: 1 and a light chain variable region (VL) comprising the amino acid sequence set forth in SEQ ID NO: 2 and/or comprising CDRs as shown in SEQ ID NO: 9, SEQ ID NO: 10 and SEQ ID NO: 11. In another example, the anti-KMA antibody has a VH comprising CDRs as shown in SEQ ID NO: 6, SEQ ID NO: 7 and SEQ ID NO: 8 and a VL comprising CDRs as shown in SEQ ID NO: 9, SEQ ID NO: 10 and SEQ ID NO: 11. Thus, in an example, the anti-KMA antibody is KM.

In another example, the anti-KMA antibody binds to or specifically binds to an epitope of KMA that is specifically bound by KM or that competes with KM for binding to KMA, wherein KM has a VH comprising the amino acid sequence set forth in SEQ ID NO: 1 and a VL comprising the amino acid sequence set forth in SEQ ID NO: 2.

KM binds an epitope of KMA located in the switch region of kappa light chain (SEQ ID NO: 3). Amino acid substitution in the epitope can increase binding affinity of KM (Hutchinson et al. 2011). Accordingly, in an example, an anti-KMA antibody according to the present disclosure competes with an antibody that binds or specifically binds a region comprising an amino acid sequence as shown in SEQ ID NO: 3 with at least one, at least two or at least three amino acid substitutions.

In another example, an anti-KMA antibody according to the present disclosure competes with an antibody that binds or specifically binds an epitope comprising an amino acid sequence as shown in SEQ ID NO: 4 with at least one, at least two or at least three amino acid substitutions. Exemplary substitutions include conservative amino acid substitutions such as those described below in Table 1. In an example, aspartic acid (Asp (D)) in SEQ ID NO: 4 is substituted with glutamic acid (Glu (E)) (SEQ ID NO: 5). Accordingly, in an example, an anti-KMA antibody according to the present disclosure competes with an antibody that binds or specifically binds an epitope comprising an amino acid sequence as shown in SEQ ID NO: 5.

TABLE 1
Exemplary substitutions.

| Original Residue | Exemplary Substitutions |
|---|---|
| Arg (R) | Lys (K) |
| Glu (E) | Asp (D) |
| Ile (I) | Leu (L); Val (V); Ala (A) |
| Leu (L) | Ile (I); Val (V); Met (M); Ala (A); Phe (F) |
| Lys (K) | Arg (R) |

In another example, an anti-KMA antibody according to the present disclosure competes with an antibody that binds or specifically binds an epitope comprising an amino acid sequence as shown in SEQ ID NO: 4. In another example, an anti-KMA antibody according to the present disclosure competes with an antibody that binds or specifically binds an epitope consisting of the amino acid sequence as shown in SEQ ID NO: 4.

Antibodies may be identified by their ability to compete for binding to KMA or a region or epitope thereof using various methods known in the art. For example, antibody binding to KMA on kappa human myeloma cell lines (KHMCL) such as KMS-11, KMS-26 and JJN3 can be assessed (Asvadi et al. 2015). In this procedure, an anti-KMA antibody such as KM is conjugated with biotin using established procedures (Hofmann K, et al. (1982) Biochemistry 21:978-84). Antibodies are then evaluated by their capacity to compete with the binding of the biotinylated KM antibody to KMA on KHMCL cells. The binding of biotinylated KM to KHMCL cells may be assessed by the addition of fluorescein-labelled streptavidin which will bind to biotin on the labelled antibody. Fluorescence staining of cells is then quantified by flow cytometry, and the competitive effect of antibodies expressed as a percentage of the fluorescence levels obtained in the absence of the competitor.

In another example, the anti-KMA antibody has a VH comprising the CDRs as shown in SEQ ID NO: 6, SEQ ID NO: 7 and SEQ ID NO: 8 and a VL. In another example, the anti-KMA antibody has a VH and a VL comprising CDRs as shown in SEQ ID NO: 9, SEQ ID NO: 10 and SEQ ID NO: 11. In another example, the anti-KMA antibody has a VH comprising CDRs as shown in SEQ ID NO: 6, SEQ ID NO: 7 and SEQ ID NO: 8 and a VL comprising CDRs as shown in SEQ ID NO: 9, SEQ ID NO: 10 and SEQ ID NO: 11.

In another example, the anti-KMA antibody has a VH comprising CDRs as shown in SEQ ID NO: 6, SEQ ID NO: 7 SEQ ID NO: 8 and an amino acid sequence at least 90%, at least 95%, at least 98% or at least 99% identical to SEQ ID NO: 1. In another example, the anti-KMA antibody has a VL comprising CDRs as shown in SEQ ID NO: 9, SEQ ID NO: 10, SEQ ID NO: 11 and an amino acid sequence at least 90%, at least 95%, at least 98% or at least 99% identical to SEQ ID NO: 2. In another example, the anti-KMA antibody has a VH comprising CDRs as shown in SEQ ID NO: 6, SEQ ID NO: 7 SEQ ID NO: 8, an amino acid sequence at least 90%, at least 95%, at least 98% or at least 99% identical to SEQ ID NO: 1 and a VL comprising CDRs as shown in SEQ ID NO: 9, SEQ ID NO: 10, SEQ ID NO: 11 and an amino acid sequence at least 90%, at least 95%, at least 98% or at least 99% identical to SEQ ID NO: 2.

In another example, the anti-KMA antibody has a VH comprising the amino acid sequence shown in SEQ ID NO: 1 and a VL comprising the amino acid sequence shown in SEQ ID NO: 2.

In another example, the anti-KMA antibody has the CDRs shown in SEQ ID NO: 1 and SEQ ID NO: 2, wherein the CDRs are assigned using the Kabat numbering system. In another example, the anti-KMA antibody has the CDRs shown in SEQ ID NO: 1 and SEQ ID NO: 2, wherein the CDRs are assigned using the IMGT numbering system. In another example, the anti-KMA antibody has the CDRs shown in SEQ ID NO: 1 and SEQ ID NO: 2, wherein the CDRs are assigned using EU numbering system of Kabat.

In an example, the anti-KMA antibody is a naked antibody. In other examples, the anti-KMA antibody is a full-length antibody, intact antibody or whole antibody. In an example, the anti-KMA antibody is monospecific.

In another example, the anti-KMA antibody is an antigen binding fragment comprising CDRs as shown in SEQ ID NO: 6, SEQ ID NO: 7, SEQ ID NO: 8, SEQ ID NO: 9, SEQ ID NO: 10 and SEQ ID NO: 11.

In another example, the anti-KMA antibody has a VH comprising the amino acid sequence shown in SEQ ID NO: 12 or a humanised variant thereof and a VL comprising the amino acid sequence shown in SEQ ID NO: 13 or a humanised variant thereof.

In another example, the anti-KMA antibody has the CDRs shown in SEQ ID NO: 12 and SEQ ID NO: 13 or humanised variants thereof, wherein the CDRs are assigned using the Kabat numbering system. In another example, the anti-KMA antibody has the CDRs shown in SEQ ID NO: 12 and SEQ ID NO: 13 or humanised variants thereof, wherein the CDRs are assigned using the IMGT numbering system. In another example, the anti-KMA antibody has the CDRs shown in SEQ ID NO: 12 and SEQ ID NO: 13 or humanised variants thereof, wherein the CDRs are assigned using EU numbering system of Kabat.

The term “daratumumab” is used in the context of the present disclosure to refer to a CD38-directed cytolytic antibody. Other terms that can be used interchangeably with “daratumumab” can include Darzalex®. The term “Darzalex® Faspro” as used herein can refer to a combination of daratumumab, a CD38-directed cytolytic antibody, and hyaluronidase, an endoglycosidase.

The term “immunomodulatory drug” or “IMiD” is used in the context of the present disclosure to refer to molecules that have pleotropic effects including inducing direct apoptosis against malignant tumor cells, interfering with the interaction of the tumor with bone marrow stromal cells, and increasing the anti-tumor immune response.

In one example, the IMiD is selected from the group consisting of lenalidomide, pomalidomide, and thalidomide.

For example, the IMiD can be lenalidomide. In one example, the IMiD can be pomalidomide. In another example, the IMiD can be thalidomide.

The term “CRBN E3 ligase modulator” or “CELMOD” is used in the context of the present disclosure to refer to IMiD derivatives. In one example, the CELMOD is selected from the group consisting of CC-122, CC-220 (iberdomide), CC-90009, CC-92480, MDEG-541 and CC-886. This list is non-limiting and various other IMiDs and CELMoDs are known in the art (e.g. see review by Holstein et al. 2018 incorporated herein by reference).

For example, the CELMOD can be CC-122. In one example, the CELMOD can be CC-220 (iberdomide). In another example, the CELMOD can be CC-90009. In another example, the CELMOD can be CC-92480. In another example, the CELMOD can be MDEG-541. In another example, the CELMOD can be CC-886.

The term “steroid” or “corticosteroid” is used in the context of the present disclosure to refer to a class of steroid hormones that are produced in the adrenal cortex or produced synthetically, such as dexamethasone and prednisone (also referred herein as prednisolone). For example, the steroid can be dexamethasone. In another example, the steroid can be prednisone.

### Dosing Regimen

The present inventors have surprisingly shown that administering an anti-KMA antibody such as KappaMab, and particularly, in combination with an IMiD and dexamethasone is unexpectedly well tolerated with a favourable safety profile, suggesting that the dose of anti-KMA antibody can be safely increased (e.g. to at least 30 mg/kg and above). Such combinations used in a dosing regimen may be particularly advantageous in view of one or more or all of increased therapeutic potency; improved outcomes; favourable safety profile. In some examples, high doses (e.g., at least 30 mg/kg) of an anti-KMA antibody, such as KappaMab, may be used as an adjuvant therapy in combination with an IMiD or CELMOD and a steroid to increase therapeutic potency, improve patient outcomes and provide a favourable safety profile.

In an example, an anti-KMA antibody having the CDRs of KM may be used as an adjuvant therapy in combination with an IMiD or CELMOD and a steroid to increase therapeutic potency of the primary treatment. In an example, an anti-KMA antibody having the CDRs of KM may be used as an adjuvant therapy in combination with an IMiD or CELMOD and a steroid to improve patient outcomes. For example, an anti-KMA antibody having the CDRs of KM may be used as an adjuvant therapy in combination with an IMiD or CELMOD and a steroid to improve overall response rate (ORR), overall survival (OS) and/or progression free survival (PFS). In one example, an anti-KMA antibody having the CDRs of KM may be used as an adjuvant therapy in combination with an IMiD or CELMOD and a steroid to reduce minimal residual disease. In one example, an anti-KMA antibody having the CDRs of KM may be used as an adjuvant therapy in combination with an IMiD or CELMOD and a steroid to reduce adverse events. In one example, an anti-KMA antibody having the CDRs of KM may be used as an adjuvant therapy in combination with an IMiD or CELMOD and a steroid to improve duration of response. In one example, an anti-KMA antibody having the CDRs of KM may be used as an adjuvant therapy in combination with an IMiD or CELMOD and a steroid to time to next treatment.

In an example, a favourable safety profile is determined based on the number or severity of adverse events reported using Common Terminology Criteria for Adverse Events v3.0 (CTCAE). For example, an adverse event may be any unfavourable and unintended event, symptom, or disease temporally associated with the combination dosing regimen.

In an example, the adverse event may be haematologic or non-haematologic. In an example, the haematologic adverse event may be anaemia, thrombocytopenia, lymphopenia, neutropenia, febrile neutropenia or leukopenia. In an example, the non-haematologic adverse event may be a rash, hyperglycemia, fatigue, abdominal pain, constipation, diarrhoea, nausea, hypophosphatemia, infusion related reaction, peripheral neuropathy, insomnia, cramps, pain, URTI, pneumonia and cellulitis. In an example, the adverse events may be graded according to severity on a scale of one to four.

In an example, a favourable safety profile is determined based on assessment of a subject's immune cells after treatment according to the disclosure. In an example, the subject's immune cells are assessed using blood analysis tests, such as, complete blood count (CBC). CBC measures a blood sample from the subject for immune cell parameters such as red blood cells (e.g. haemoglobin and haematocrit), white blood cells (e.g. neutrophils) and platelets.

For example, the subject is assessed after administration of four to eight doses of an anti-KMA antibody of the disclosure. In an example, the subject is assessed prior to the treatment phase (i.e. prior to administration of the anti-KMA antibody). In an example, the subject can be assessed after four to eight weeks of the treatment phase. In another example, the subject's immune cells are compared with the subject's immune cells at baseline (i.e. prior to administration of the anti-KMA antibody). In an example, the subject's immune cells are monitored over time and a favourable safety profile is characterised by no significant change of the monitored cell(s). For example, the subject's immune cells can be monitored over time and, a favourable safety profile is characterised by no significant change in the subject's immune cells. In an example, a favourable safety profile is characterised by no change in the subject's immune cells after administration of four doses of an anti-KMA antibody such as KM. In an example, a favourable safety profile is characterised by no change in the subject's immune cells after administration of eight doses of an anti-KMA antibody such as KM. In an example, the immune cells are one or more or all of red blood cells (e.g. haemoglobin and haematocrit); white blood cells (e.g. neutrophils); and platelets.

In an example, anaemia can be diagnosed using haemoglobin as a parameter. For example, mild anaemia can be diagnosed under the National Cancer Institute (NCI) criteria as haemoglobin levels of 10 g/dL to a lower limit of normal. In an example, moderate anaemia can be diagnosed under the NCI criteria as haemoglobin levels between 10-8 g/dL. In an example, severe anaemia can be diagnosed under the NCI criteria as haemoglobin levels between 7.9-6.5 g/dL. In an example, life-threatening anaemia can be diagnosed under the NCI criteria as haemoglobin levels lower than 6.5 g/dL.

For example, grade one anaemia can be diagnosed under CTCAE grading as haemoglobin levels<institutional lower limit of normal (LLN)−10 g/dL; <LLN−6.2 mmol/L; <LLN−100 g/L around 10 g/dL. In an example, grade two anaemia can be diagnosed under CTCAE grading as haemoglobin levels<10.0-8.0 g/dL; <6.2-4.9 mmol/L; <100-80 g/L. In an example, grade three anaemia can be diagnosed under CTCAE grading as haemoglobin levels<8.0 g/dL; <4.9 mmol/L; <80 g/L.

In an example, thrombocytopenia can be diagnosed using platelet count as a parameter. For example, grade one thrombocytopenia can be diagnosed as a platelet count of 75 to <100×10⁹/L. In an example, grade two thrombocytopenia can be diagnosed as a platelet count of 50 to <75×10⁹/L. In an example, grade three thrombocytopenia can be diagnosed as a platelet count of 25 to <5 0×10⁹/L. In an example, grade four thrombocytopenia can be diagnosed as a platelet count of <25×10⁹/L. It will be understood that platelet thresholds are based on those clinically relevant for chemotherapy and grades 2 to 4 CTCAE V5.0.

In an example, neutropenia can be diagnosed using absolute neutrophil count as a parameter. For example, grade one neutropenia can be diagnosed as an absolute neutrophil count (ANC) around 1,500 ANC/μL. In an example, grade two neutropenia can be diagnosed as an absolute neutrophil count between 1,500-1,000 ANC/μL. In an example, grade three neutropenia can be diagnosed as an absolute neutrophil count between 1,000-500 ANC/μL. In an example, grade four neutropenia can be diagnosed as an absolute neutrophil count lower than 500 ANC/μL.

In an example, the dose of an anti-KMA antibody disclosed herein is at least 30 mg/kg. In another example, the dose of the anti-KMA antibody ranges from about 30 mg/kg to about 90 mg/kg. In one example, the dose of the anti-KMA antibody ranges from 30 mg/kg to 90 mg/kg. In another example, the dose of the anti-KMA antibody ranges from about 30 mg/kg to about 60 mg/kg. In one example, the dose of the anti-KMA antibody ranges from 30 mg/kg to 60 mg/kg. In another example, the dose of the anti-KMA antibody ranges from about 30 mg/kg to about 45 mg/kg. In one example, the dose of the anti-KMA antibody ranges from 30 mg/kg to 45 mg/kg. In another example, the dose of the anti-KMA antibody is 30 mg/kg. In another example, the dose of the anti-KMA antibody is 45 mg/kg. In another example, the dose of the anti-KMA antibody is 60 mg/kg. In another example, the dose of the anti-KMA antibody is 90 mg/kg.

As will be appreciated by those of skill in the art, the appropriate dose of the IMiD or the CELMOD will depend on the IMiD or the CELMOD being administered. Exemplary general doses of the IMiD or the CELMOD range from about 5 mg to about 300 mg. For example, the dose of the IMiD or the CELMOD range from 5 mg to 300 mg. In one example, the dose of the IMiD or the CELMOD ranges from about 10 mg to about 200 mg. For example, the dose of the IMiD or the CELMOD ranges from 10 mg to 200 mg. In an example, the dose of the IMiD or the CELMOD ranges from about 10 mg to about 25 mg. For example, the dose of the IMiD or the CELMOD ranges from 10 mg to 25 mg. In another example, the dose of the IMiD or the CELMOD is about 10 mg. In one example, the dose of the IMiD or the CELMOD is 10 mg. In another example, the dose of the IMiD or the CELMOD is about 15 mg. In one example, the dose of the IMiD or the CELMOD is 15 mg. In another example, the dose of the IMiD or the CELMOD is about 25 mg. In one example, the dose of the IMiD or the CELMOD is 25 mg.

In an example, the dose of lenalidomide ranges from about 5 mg to about 300 mg. For example, the dose of lenalidomide ranges from 5 mg to 300 mg. In one example, the dose of lenalidomide ranges from about 10 mg to about 200 mg. For example, the dose of lenalidomide ranges from 10 mg to 200 mg. In an example, the dose of lenalidomide ranges from about 10 mg to about 25 mg. For example, the dose of lenalidomide ranges from 10 mg to 25 mg. In another example, the dose of lenalidomide is about 10 mg. In one example, the dose of lenalidomide is 10 mg. In another example, the dose of lenalidomide is about 15 mg. In one example, the dose of lenalidomide is 15 mg. In another example, the dose of lenalidomide is about 25 mg. In one example, the dose of lenalidomide is 25 mg.

In an example, the dose of pomalidomide ranges from about 1 mg to about 5 mg. For example, the dose of pomalidomide ranges from 1 mg to 5 mg. In one example, the dose of pomalidomide ranges from 1 mg to about 4 mg. In another example, the dose of pomalidomide is about 4 mg. In one example, the dose of pomalidomide is 3 mg. In another example, the dose of pomalidomide is 2 mg. In one example, the dose of pomalidomide is 1 mg.

Appropriate IMiD or CELMOD dosing can also be obtained from prescribing information. For example, lenalidomide can be administered at 25 mg for newly diagnosed subjects. In one example, lenalidomide can be initially administered at 10 mg before being increased to 15 mg or 25 mg if well tolerated. In another example, pomalidomide can be administered at 4 mg for newly diagnosed subjects. In one example, pomalidomide can be administered at 3 mg for subjects over the age of 75 years.

The appropriate dose of the steroid will depend on the steroid being administered. Exemplary general doses of a steroid range from about 5 mg to about 300 mg. For example, the dose of the steroid ranges from 5 mg to 300 mg. In one example, the dose of the steroid ranges from about 10 mg to about 100 mg. In one example, the dose of the steroid ranges from 10 mg to 100 mg. In an example, the dose of the steroid ranges from about 40 mg to about 60 mg. In an example, the dose of the steroid ranges from 40 mg to 60 mg. In another example, the dose of the steroid is about 40 mg. In one example, the dose of the steroid is 40 mg. In another example, the dose of the steroid is about 60 mg. In one example, the dose of the steroid is 60 mg. In another example, the dose of the steroid is about 25 mg. In one example, the dose of the steroid is 25 mg.

In an example, the dose of dexamethasone ranges from about 5 mg to about 300 mg. In an example, the dose of dexamethasone ranges from 5 mg to 300 mg. In one example, the dose of dexamethasone ranges from about 10 mg to about 100 mg. In one example, the dose of dexamethasone ranges from 10 mg to 100 mg. In an example, the dose of dexamethasone ranges from about 40 mg to about 60 mg. In an example, the dose of dexamethasone ranges from 40 mg to 60 mg. In another example, the dose of dexamethasone is about 40 mg. In one example, the dose of dexamethasone is 40 mg. In another example, the dose of dexamethasone is about 60 mg. In one example, the dose of dexamethasone is 60 mg. In another example, the dose of dexamethasone is about 25 mg. In one example, the dose of dexamethasone is 25 mg.

Appropriate steroid dosing can also be obtained from prescribing information and, if required, amended based on clinical response. For example, dexamethasone can be administered at 40 mg and reduced if there are specific side effects. In another example, dexamethasone can be administered at 20 mg for subjects over the age of 75 years.

### Treatment Phase

Treatment phase according to the present disclosure comprises administering an anti-KMA antibody, such as KM, an IMiD or a CELMOD and a steroid. In an example, the treatment phase may comprise administration of an anti-KMA antibody about once per week, an IMiD or a CELMOD about once per day, and a steroid about once per week. In an example, the treatment phase comprises administration of the anti-KMA antibody once per week (i.e. once weekly). In an example, the treatment phase comprises administration of the anti-KMA antibody weekly for at least four, at least five, at least six, at least seven, at least eight, at least nine or at least 10 weeks. In an example, the treatment phase comprises administration of the anti-KMA antibody weekly for about eight weeks. In these examples, weekly administration can be referred to as a treatment dose (i.e., treatment phase). The dose of the anti-KMA antibody may be increased or decreased throughout the treatment phase as required. For example, the treatment phase may comprise administration of KM once per week. In one example, the treatment phase may comprise administration of a IMiD or a CELMOD about once per day for about 21 days, followed by one week with no IMiD or CELMOD administration.

In one example, the dose of the anti-KMA antibody may be stopped for a period of time as required (e.g., when trying to resolve a severe adverse event, or as a result of subject non-compliance). In another example, the dose of the anti-KMA antibody may be delayed for a period of time as required. In another example, the dose of the anti-KMA antibody may be reduced for a period of time as required.

In an example, the treatment phase may comprise administration of the IMiD or the CELMOD once per day (i.e. once daily). In another example, the treatment phase may comprise administration of the IMiD or the CELMOD once every second day. In another example, the treatment phase may comprise administration of the IMiD or the CELMOD once every third day. The dose of the IMiD or the CELMOD may be increased or decreased throughout the treatment phase as required. For example, the treatment phase may comprise administration of lenalidomide once per day. In another example, the treatment phase may comprise administration of pomalidomide once per day.

In an example, the treatment phase may comprise administration of the steroid once per week (i.e. weekly). In another example, the treatment phase may comprise administration of the steroid once every second week (i.e. fortnightly). In another example, the treatment phase may comprise administration of the steroid once every third week. In another example, the treatment phase may comprise administration of the steroid once every fourth week. The dose of the steroid may be increased or decreased throughout the treatment phase as required. For example, the treatment phase may comprise administration of dexamethasone once per week.

In an example, the duration of the treatment phase is at least four, at least five, at least six, at least seven, at least eight, at least nine, at least 10, at least 11 or at least 12 weeks. In an example, the duration of the treatment phase is at least six weeks to at least 10 weeks. In another example, the duration of the treatment phase is at least eight weeks.

### Induction Phase

In certain examples, methods of the present disclosure comprise an induction phase. In an example, the induction phase precedes a treatment phase.

In an example, an induction phase precedes administration of an anti-KMA antibody to the subject. In an example, the induction phase comprises administration of the IMiD or the CELMOD with the intention of reducing multiple myeloma cell proliferation and improving the response to an anti-KMA antibody. In an example, the induction phase may comprise administration of the IMiD or the CELMOD once per day (i.e. once daily). In another example, the induction phase may comprise administration of the IMiD or the CELMOD once every second day. In another example, the induction phase may comprise administration of the IMiD or the CELMOD once every third day. The dose of the IMiD or the CELMOD may be increased or decreased throughout the induction phase as required.

In an example, the induction phase may comprise administration of the IMiD or the CELMOD at least one, at least two, at least three, at least four or at least five weeks prior to the start of the treatment phase. In an example, the induction phase may comprise administration of the IMiD or the CELMOD for at least one week prior to the start of the treatment phase. For example, the induction phase may comprise administration of lenalidomide once per day for at least one week. For example, the induction phase may comprise administration of pomalidomide once per day for at least one week.

In an example, the induction phase may comprise administration of the steroid to help reduce malignant cell proliferation to inhibit cytokine production and reduce the activity of nuclear factor kappa B, which are required for malignant plasma cell survival. In an example, the induction phase may comprise administration of the steroid twice per week. In an example, the induction phase may comprise administration of the steroid once per week (i.e. weekly). In another example, the induction phase may comprise administration of the steroid once every second week (i.e. fortnightly). The dose of the steroid may be increased or decreased throughout the induction phase as required.

In an example, the induction phase may comprise administration of the steroid at least one, at least two, at least three, at least four or at least five weeks prior to the start of the treatment phase. In an example, the induction phase may comprise administration of the steroid for at least one week prior to the start of the treatment phase. For example, the induction phase may comprise administration of dexamethasone at least one week prior to the start of the treatment phase.

In an example, the induction phase may comprise administration of the IMiD or the CELMOD about once per day, and the steroid about once per week at least one week prior to the start of the treatment phase.

In an example, the duration of the induction phase is at least one, at least two, at least three or at least four weeks. For example, the duration of the induction phase is at least one week.

### Maintenance Phase

In certain examples, methods of the present disclosure comprise a maintenance phase. In an example, the maintenance phase follows a treatment phase. In an example, the maintenance phase comprises administration of an anti-KMA antibody once every second week (i.e. fortnightly). In an example, the maintenance phase comprises administration of the anti-KMA antibody once per month (i.e. monthly). For example, the maintenance phase comprises administration of the anti-KMA monthly for at least one, at least two, at least three, at least four, at least five, at least six, at least seven or at least eight weeks. In these examples, monthly administration can be referred to as a maintenance dose (i.e., maintenance phase; also known as a follow up phase). In these examples, administration of the anti-KMA antibody during the maintenance phase can serve to modulate, enhance, increase or augment the subject's response to any or all lines of therapy administered during the treatment phase. In one example, administration of the anti-KMA antibody during the maintenance phase can serve to modulate, enhance, increase or augment the subject's response to the IMID, CELMOD and/or steroid administered during the treatment phase. In these examples, the anti-KMA can serve as an adjuvant and the maintenance phase may therefrom be referred to as an adjuvant or adjunct phase or an adjuvant or adjunct therapy.

In an example, the maintenance phase may comprise administration of the IMiD or the CELMOD once per day (i.e. once daily). In another example, the maintenance phase may comprise administration of the IMiD or the CELMOD once every second day. In another example, the maintenance phase may comprise administration of the IMiD or the CELMOD once every third day. The dose of the IMiD or the CELMOD may be increased or decreased throughout the maintenance phase as required. For example, the maintenance phase may comprise administration of lenalidomide once per day. For example, the maintenance phase may comprise administration of pomalidomide once per day.

In one example, the dose of the IMiD may be stopped for a period of time as required (e.g., when trying to resolve a severe adverse event, such as a cytopenia). In another example, the dose of the IMiD may be delayed for a period of time as required.

In an example, the maintenance phase may comprise administration of the steroid once per week (i.e. weekly). In another example, the maintenance phase may comprise administration of the steroid once every second week (i.e. fortnightly). In another example, the maintenance phase may comprise administration of the steroid once every third week. In another example, the maintenance phase may comprise administration of the steroid once every fourth week. The dose of the steroid may be increased or decreased throughout the maintenance phase as required. For example, the maintenance phase may comprise administration of dexamethasone once per week.

In an example, the maintenance phase may comprise administration of the anti-KMA antibody about once per month, the IMiD or the CELMOD about once per day, and the steroid about once per week.

In an example, the duration of the maintenance phase is at least 8, at least 9, at least 10, at least 11, at least 12, at least 13 or at least 14 weeks. For example, the duration of the maintenance phase is at least 12 weeks. In another example, any of the aforementioned maintenance phases may be continued until disease progression (i.e. indefinitely).

### Administration

The above referenced dosing regimen requires administration via various routes. Exemplary routes of administration include intravenous administration as a bolus, continuous infusion over a period of time, or oral administration. Those of skill in the art will appreciate that the most appropriate route of administration will be largely dependent on the treatment being administered. For example, an anti-KMA antibody can be administered intravenously.

In an example, the anti-KMA antibody is administered intravenously via a bolus. In an example, the anti-KMA antibody is administered intravenously via continuous infusion. In an example, intravenous infusion of an anti-KMA antibody lasts about one to two hours.

In an example, the IMiD or the CELMOD is administered orally. For example, these treatments may be administered orally via a tablet. In an example, the steroid is administered intravenously.

In an example, the anti-KMA antibody, the IMiD or the CELMOD and the steroid are administered as separate compositions. For example, the anti-KMA antibody, the IMiD or the CELMOD and the steroid can be administered sequentially. In this example, administration of the anti-KMA antibody, IMiD or CELMOD and steroid is carried out over a defined time period (usually hours or days). In an example, the period between sequential administration can be several days up to several weeks, provided that there is still sufficient levels of the first therapeutic to provide or add to the therapeutic benefit of the second or third therapeutic when it is administered.

In an example, the IMiD or the CELMOD and the steroid are administered via the same route. For example, both the IMiD or the CELMOD and the steroid can be administered orally. In an example, the IMiD or the CELMOD can be given as an oral drug at about 25 mg per day, once daily. For example, lenalidomide can be given orally at about 25 mg, once per day. For example, pomalidomide can be given orally at about 4 mg, once per day. In an example, the steroid can be given as an oral drug at about 40 mg per day, once weekly. For example, dexamethasone can be given orally at about 25 mg, once per day.

In an example, dosing can be adjusted based on clinical evaluation or, if appropriate, prescribing information. Exemplary clinical evaluation may include physical examination, assessment of haematological toxicity (e.g. determining anaemia, neutropenia or thrombocytopenia).

### Methods of Treatment

In one example, the present disclosure relates to a method of treating multiple myeloma in a subject in need thereof, the method comprising administering to the subject a treatment which comprises an anti-KMA antibody, an immunomodulatory drug (IMiD) or a cereblon E3 ligase modulator (CELMOD), and a steroid. For example, the methods can comprise administering the above referenced dosing regimen.

As used herein, the terms “treating”, “treat” or “treatment” include administering a therapeutically effective amount of an anti-KMA antibody, an IMiD or a CELMOD, and a steroid to reduce or delay the onset or progression of multiple myeloma, or to reduce or eliminate at least one symptom of multiple myeloma.

The terms “multiple myeloma” or “myeloma” or “MM” are used in the context of the present disclosure to refer to cancer of plasma cells. In the context of the present disclosure, these terms encompasses secretory myeloma, non-secretory myeloma, light chain only myeloma, smouldering myeloma and related pathologies. Exemplary related pathologies include plasmacytoma, amyloidosis, monoclonal gammopathy of undetermined significance.

In an example, the multiple myeloma is kappa type myeloma. For example, the multiple myeloma expresses KMA. In another example, the multiple myeloma expresses kappa light chain. In an example, the multiple myeloma expresses IgG Kappa. In a further example, the multiple myeloma expresses IgA Kappa.

Subjects with multiple myeloma can be characterised into various subject populations. Exemplary populations are described in Rajkumar et al. 2011.

In an example, a subject's multiple myeloma can be characterised as progressive disease (Rajkumar et al. 2011). Put another way, the methods of the present disclosure relate to the treatment of progressive multiple myeloma in a subject. Exemplary indicators of “progressive disease” include an increase of about 25% from the lowest response value in any one of the following: Serum M-component (e.g., absolute increase> or equal to 0.5 g/dL) and/or Urine M-component (e.g., absolute increase must be > or equal to 200 mg/24 hr). Other exemplary indicators include definite development of new bone lesions or soft tissue plasmacytomas or definite increase in the size of existing bone lesions or soft tissue plasmacytomas; development of hypercalcemia (e.g., corrected serum calcium>11.5 mg/dL) that can be attributed solely to the multiple myeloma. In an example, the subject's multiple myeloma has relapsed and is characterised as progressive disease. In this example, the subject's multiple myeloma can also be refractory to therapy.

In another example, the subject has primary refractory myeloma. “Primary refractory myeloma” is used to refer to disease that is nonresponsive in subjects who have never achieved a minimal response or better with any therapy.

In another example, the subject has refractory myeloma. The term “refractory myeloma” is used to refer to disease that is nonresponsive while on primary or salvage therapy, or progresses within 60 days of last therapy. In an example, a subjects multiple myeloma is refractory to an anti-cancer therapy. The term “refractory” is used in this context to refer to a line of anti-cancer therapy that is no longer therapeutically effective against a subject's multiple myeloma.

In an example, the subject's multiple myeloma has relapsed. “Relapsed myeloma” is used to refer to previously treated myeloma that progresses and requires the initiation of salvage therapy but does not meet criteria for either “primary refractory myeloma” or “refractory myeloma”.

In an example, a subject treated by the methods of the present disclosure can be refractory to at least one proteasome inhibitor. For example, a subject can be refractory to bortezomib. A “line of therapy” is defined as one or more cycles of a planned treatment program. This may consist of one or more planned cycles of single-agent therapy or combination therapy, as well as a sequence of treatments administered in a planned manner. For example, a planned treatment approach of induction therapy followed by autologous stem cell transplantation, followed by maintenance is considered one line of therapy.

In another example, subjects are refractory to at least two prior lines of therapy. In another example, a subject may be refractory to at least three, at least four, at least five or at least six prior lines of therapy. In this example, at least one line of therapy may be lenalidomide.

In another example, the subject has relapsed and refractory myeloma. “Relapsed and refractory myeloma” is used to refer to disease that is nonresponsive while on salvage therapy, or progresses within 60 days of last therapy in subjects who have achieved minimal response (MR) or better at some point previously before then progressing in their disease course.

In an example, the multiple myeloma treated according to the present disclosure is characterised as stable disease at the time of first administration. Put another way, subjects can be in plateau phase at the time of first administration. Exemplary criteria for stable disease can include stabilization of the M-protein without further tumour regression despite continued treatment, few or no symptoms from myeloma and/or no blood transfusion requirement (Blade et al. 1998).

Subjects treated according to the methods of the present disclosure have multiple myeloma or a related pathology encompassed by the present disclosure.

In an example, a subject treated according to the present disclosure has received at least one line of prior therapy for their multiple myeloma. For example, a subject's multiple myeloma can have relapsed. In another example, a subject has received at least two, at least three, at least four, at least five or at least six prior lines of therapy. In these examples, a subject can have achieved at least a minimal response (e.g., about 25% reduction in M protein) to their most recent line of therapy.

In another example, a subject has serum kappa free light chain levels less than about 350 mg/ml. In another example, a subject has serum kappa free light chain levels less than about 300 mg/ml. In another example, a subject has serum kappa free light chain levels less than about 275 mg/ml. In another example, a subject has serum kappa free light chain levels less than about 250 mg/ml.

In another example, the methods of the present disclosure also relate to treating multiple myeloma in subjects with high serum cytokine levels. For example, the methods of the present disclosure relate to treating multiple myeloma in a subject, the method comprising selecting a subject who has high serum levels of one or more of the following factors relative to control serum levels: hepatocyte growth factor (HGF), macrophage inhibitory factor (MIF), CCL27, G-CSF, CXCL9, and CXCL10; and administering to the subject an anti-KMA antibody. In one aspect of the examples herein, serum analyte levels described herein are determined by immunoassay.

In an example, a high serum level of HGF is above about 0.5 ng/ml. In an example, a high serum level of HGF is above about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml or about 1.5 ng/ml. In another example, a high serum level of HGF is at least about 1.6 ng/ml. In another example, a high serum level of HGF is at least 1.6 ng/ml.

In another example, a high serum level of MIF is above about 5000 pg/ml. In another example, a high serum level of MIF is above about 5200 pg/ml, about 5400 pg/ml, about 5600 pg/ml, about 5800 pg/ml, about 6000 pg/ml, about 6200 pg/ml, about 6400 pg/ml, about 6600 pg/ml, about 6800 pg/ml or about 7200 pg/ml. In one example, a high serum level of MIF is above 5000 pg/ml.

In another example, a high serum level of CCL27 is above about 500 pg/ml. In another example, a high serum level of CCL27 is above about 600 pg/ml, about 700 pg/ml, about 800 pg/ml, about 900 pg/ml, about 1000 pg/ml, about 1100 pg/ml, about 1200 pg/ml, about 1300 pg/ml, about 1400 pg/ml or about 1500 pg/ml. In one example, a high serum level of CCL27 is above 500 pg/ml.

In another example, a high serum level of G-CSF is above about 55 pg/ml. In another example, a high serum level of G-CSF is above about 65 pg/ml, about 75 pg/ml, about 85 pg/ml, about 95 pg/ml, about 105 pg/ml, about 115 pg/ml, about 125 pg/ml, about 135 pg/ml, about 145 pg/ml or about 155 pg/ml. In one example, a high serum level of G-CSF is above 55 pg/ml.

In another example, a high serum level of CXCL9 is above about 550 pg/ml. In another example, a high serum level of CXCL9 is above about 600 pg/ml, about 650 pg/ml, about 700 pg/ml, about 750 pg/ml, about 800 pg/ml, about 850 pg/ml, about 900 pg/ml, about 950 pg/ml, about 1000 pg/ml or about 1050 pg/ml. In one example, a high serum level of CXCL9 is above 550 pg/ml.

In another example, a high serum level of CXCL10 is above about 850 pg/ml. In another example, a high serum level of CXCL10 is above about 900 pg/ml, about 950 pg/ml, about 1000 pg/ml, about 1050 pg/ml, about 1100 pg/ml, about 1150 pg/ml, about 1200 pg/ml, about 1250 pg/ml, about 1300 pg/ml or about 1350 pg/ml. In another example, a high serum level of CXCL10 is above 850 pg/ml.

High serum cytokine levels are determined in a sample obtained from the subject.

### Additional Steps

In another example, the methods of the present disclosure further comprise administering one or more additional agents or treatments. For example, anti-cancer agents, proteasome inhibitors or autologous stem cell transplantation. Exemplary anti-cancer agents include histone deacetylase inhibitors such as panobinostat or vorinostat, antibodies such as elotuzumab, daratumumab, isatuximab or anti-PD1 antibodies such as pembrolizumab, nivolumab and atezolizumab. Exemplary proteasome inhibitors include marizomib, oprozomib, epoxomicin, salinosporamide A, carfilzomib, ixazomib and bortezomib. Autologous stem cell transplantation is the use of healthy blood stem cells from the subject's body to replace diseased or damaged bone marrow.

In an example, at least one additional agent is administered. For example, bortezomib can be administered. In other examples, at least two, at least three, at least four, at least five, at least six additional agents may be administered.

In an example, at least one additional agent is administered. For example, daratumumab can be administered. In one embodiment, daratumumab is administered as a primary treatment and an anti-KMA antibody, such as KappaMab, may be used as an adjuvant therapy in combination with an IMiD or CELMOD and a steroid as provided herein. In one embodiment, daratumumab is administered as a primary treatment along with the combination treatment comprising an anti-KMA antibody (e.g., KappaMab), an IMiD or CELMOD and a steroid as provided herein.

In an example, daratumumab is administered intravenously via a bolus. In an example, daratumumab is administered intravenously via continuous infusion. In an example, intravenous infusion of daratumumab follows the infusion rates found in Table 2.

TABLE 2
Infusion Rates for Daratumumab Administration

| | | | |
|---|---|---|---|
| Dilution | Initial Rate | Rate | Maximum |
| Volume | (first hour) | Increment^{a} | Rate |

Week 1 infusion

| | | | | |
|---|---|---|---|---|
| Option 1 (single dose | | | | |
| infusion) | | | | |
| Week 1 Day 1 (16 | 1,000 ml | 50 ml/hour | 50 ml/hour | 200 ml/hour |
| mg/kg) | | | every hour | |
| Option 2 (split dose | | | | |
| infusion) | | | | |
| Week 1 Day 1 (8 | 500 ml | 50 ml/hour | 50 ml/hour | 200 ml/hour |
| mg/kg) | | | every hour | |
| Week 1 Day 2 (8 | 500 ml | 50 ml/hour | 50 ml/hour | 200 ml/hour |
| mg/kg) | | | every hour | |
| Week 2 (16 mg/kg)^{b} | 500 ml | 50 ml/hour | 50 ml/hour | 200 ml/hour |
| | | | every hour | |
| Week 3 onwards (16 | 500 ml | 100 ml/hour | 50 ml/hour | 200 ml/hour |
| mg/kg)^{c} | | | every hour | |

^{a}Consider incremental escalation of the infusion rate only in the absence of infusion-related reactions.
^{b}Use a dilution volume of 500 mL for the 16 mg/kg dose only if there were no infusion-related reactions the previous week. Otherwise, use a dilution volume of 1,000 mL.
^{c}Use a modified initial rate (100 mL/hour) for subsequent infusions (i.e. Week 3 onwards) only if there were no infusion-related reactions during the previous infusion. Otherwise, continue to use instructions indicated in the table for the Week 2 infusion rate.

In an example, dosing for daratumumab or the daratumumab/hyaluronidase combination can be adjusted based on clinical evaluation or, if appropriate, prescribing information. Exemplary clinical evaluation may include physical examination, and assessment of haematological toxicity (e.g. determining anaemia, neutropenia or thrombocytopenia). Adverse reactions that can be monitored include upper respiratory infection, neutropenia, infusion-related reactions, thrombocytopenia, diarrhea, constipation, anemia, peripheral sensory neuropathy, fatigue, peripheral edema, nausea, cough, pyrexia, dyspnea, and asthenia. In some cases, antiviral prophylaxis is initiated to prevent herpes zoster reactivation within 1 week after starting daratumumab or the daratumumab/hyaluronidase combination and continued for 3 months following the end of treatment.

Appropriate daratumumab or daratumumab/hyaluronidase combination dosing for any use provided herein can also be obtained from respective prescribing information. In one embodiment, daratumumab or the daratumumab/hyaluronidase combination is used as monotherapy. In another embodiment, daratumumab or the daratumumab/hyaluronidase combination is used in combination with an IMiD and a steroid. Appropriate IMiD or CELMOD dosing can also be obtained from prescribing information.

In an example, daratumumab or the daratumumab/hyaluronidase combination is used as monotherapy or in combination with an IMiD and a steroid in accordance with the dosing schedule found in Table 3. In one embodiment, the IMiD is lenalidomide. In one embodiment, the IMiD is pomalidomide. In one embodiment, the steroid is dexamethasone. In one embodiment, the steroid is low-dose dexamethasone. In embodiment, the dose of daratumumab is 16 mg/kg actual body weight of a subject to be treated. In one embodiment, the daratumumab is administered as an intravenous infusion. In one embodiment, the subject has relapsed/refractory multiple myeloma and the 16 mg/kg daratumumab is administered as a monotherapy according to Table 3. In embodiment, the dose of the daratumumab/hyaluronidase combination is 1800 mg daratumumab/30,000 units hyaluronidase. In one embodiment, the daratumumab/hyaluronidase combination is administered subcutaneously over approximately 3-5 minutes. In one embodiment, the subject has relapsed/refractory multiple myeloma and the 1800 mg daratumumab/30,000 units hyaluronidase is administered as a monotherapy according to Table 3.

In one embodiment, the subject has been newly diagnosed with multiple myeloma and is ineligible for autologous stem cell transplant and the daratumumab is administered in combination with lenalidomide and dexamethasone (low-dose). Further to this embodiment, the lenalidomide can be administered at 25 mg once daily orally on days 1-21 of a repeated 28-day (4 week) cycle, while the dexamethasone is administered as low dose oral or intravenous dexamethasone 40 mg/week (or a reduced dose of 20 mg/week for patients>75 years or body mass index [BMI]<18.5). In some cases, the 16 mg/kg daratumumab is administered according to Table 3. On daratumumab infusion days, the dexamethasone dose can be given as a pre-infusion medication. In some cases, at least 30 mg/kg of an anti-KMA antibody (e.g., an anti-KMA antibody having the CDRs of KM) can also be administered.

In one embodiment, the subject has multiple myeloma and has received at least one prior therapy and the daratumumab is administered in combination with lenalidomide and dexamethasone (low-dose). Further to this embodiment, the lenalidomide can be administered at 25 mg once daily orally on days 1-21 of a repeated 28-day (4 week) cycle, while the dexamethasone is administered as low dose oral or intravenous dexamethasone 40 mg/week (or a reduced dose of 20 mg/week for patients>75 years or BMI<18.5). In some cases, on daratumumab infusion days, 20 mg of the dexamethasone dose can be given as a pre-infusion and the remainder given the day after infusion. In some cases, on daratumumab infusion days, 20 mg of the dexamethasone dose can be given as a pre-infusion and the remainder given the day after infusion. For subjects on a reduced dexamethasone dose, the entire 20 mg dose can be given as a pre-infusion medication. 16 mg/kg daratumumab is administered according to Table 3. In some cases, at least 30 mg/kg of an anti-KMA antibody (e.g., an anti-KMA antibody having the CDRs of KM) can also be administered.

In one embodiment, the subject has been newly diagnosed with multiple myeloma and is ineligible for autologous stem cell transplant and the daratumumab/hyaluronidase combination is administered in combination with lenalidomide and dexamethasone. Further to this embodiment, the lenalidomide can be administered at 25 mg once daily orally on days 1-21 of a repeated 28-day (4 week) cycle, while the dexamethasone is administered as oral or intravenous dexamethasone 40 mg/week (or a reduced dose of 20 mg/week for patients>75 years or body mass index [BMI]<18.5). In some cases, the 1800 mg daratumumab/30,000 units hyaluronidase combination is administered subcutaneously over 3-5 minutes according to Table 3. In some cases, at least 30 mg/kg of an anti-KMA antibody (e.g., an anti-KMA antibody having the CDRs of KM) can also be administered.

In one embodiment, the subject has relapsed/refractory multiple myeloma and the daratumumab/hyaluronidase combination is administered in combination with lenalidomide and dexamethasone. Further to this embodiment, the lenalidomide can be administered at 25 mg once daily orally on days 1-21 of a repeated 28-day (4 week) cycle, while the dexamethasone is administered as oral or intravenous dexamethasone 40 mg/week (or a reduced dose of 20 mg/week for patients>75 years or body mass index [BMI]<18.5). In some cases, the 1800 mg daratumumab/30,000 units hyaluronidase combination is administered subcutaneously over 3-5 minutes according to Table 3. In some cases, at least 30 mg/kg of an anti-KMA antibody (e.g., an anti-KMA antibody having the CDRs of KM) can also be administered.

In one embodiment, the subject has relapsed/refractory multiple myeloma and the daratumumab is administered in combination with pomalidomide and dexamethasone (low-dose). Further to this embodiment, the pomalidomide can be administered at 4 mg once daily orally on days 1-21 of a repeated 28-day (4 week) cycle, while the dexamethasone is administered as low dose oral or intravenous dexamethasone 40 mg/week (reduced dose of 20 mg/week for patients>75 years or BMI<18.5). In some cases, on daratumumab infusion days, 20 mg of the dexamethasone dose can be given as a pre-infusion and the remainder given the day after infusion. For subjects on a reduced dexamethasone dose, the entire 20 mg dose can be given as a pre-infusion medication. 16 mg/kg daratumumab is administered according to Table 3. In some cases, at least 30 mg/kg of an anti-KMA antibody (e.g., an anti-KMA antibody having the CDRs of KM) can also be administered.

In one embodiment, the subject has relapsed/refractory multiple myeloma and the daratumumab/hyaluronidase combination is administered in combination with pomalidomide and dexamethasone. Further to this embodiment, the pomalidomide can be administered at 4 mg once daily orally on days 1-21 of a repeated 28-day (4 week) cycle, while the dexamethasone is administered as low dose oral or intravenous dexamethasone 40 mg/week (reduced dose of 20 mg/week for patients>75 years). In some cases, the 1800 mg daratumumab/30,000 units hyaluronidase combination is administered subcutaneously over 3-5 minutes according to Table 3. In some cases, at least 30 mg/kg of an anti-KMA antibody (e.g., an anti-KMA antibody having the CDRs of KM) can also be administered.

In one embodiment, the subject has multiple myeloma and has received at least one to three prior lines of therapy and the daratumumab/hyaluronidase combination is administered in combination with carfilzomib and dexamethasone (4-week cycle). Carfilzomib can be administered intravenously at a dose of 20 mg/m2 on Cycle 1 Day 1 and if a dose of 20 mg/m2 was tolerated, carfilzomib was administered at a dose of 70 mg/m2 as a 30-minute IV infusion, on Cycle 1 Day 8 and Day 15, and then Day 1, 8 and 15 of each cycle. Dexamethasone 40 mg can be administered orally or intravenously (or a reduced dose of 20 mg per week for patients≥75 years or BMI<18.5). The daratumumab/hyaluronidase combination can be administered subcutaneously according to Table 3. The 1800 mg daratumumab/30,000 units hyaluronidase combination can be administered subcutaneously once weekly from Weeks 1 to 8, once every 2 weeks from Weeks 9 to 24 and once every 4 weeks starting with Week 25 until disease progression or unacceptable toxicity. In some cases, at least 30 mg/kg of an anti-KMA antibody (e.g., an anti-KMA antibody having the CDRs of KM) can also be administered.

TABLE 3
Daratumumab or daratumumab/hyaluronidase combination
dosing schedule in combination with IMiD (e.g.,
lenalidomide or pomalidomide) (4-week cycle) or
proteasome inhibitor (i.e., carfilzomib) and a steroid (e.g.,
low-dose dexamethasone) and for monotherapy.

| Weeks | Schedule |
|---|---|
| Weeks 1 to 8 | Weekly (total of 8 doses) |
| Weeks 9 to 24^{a} | Every two weeks (total of 8 doses) |
| Week 25 onwards until | Every four weeks |
| disease progression^{b} | |

^{a}First dose of the every-2-week dosing schedule is given at week 9.
^{b}First dose of the every-4-week dosing schedule is given at week 25.

In another embodiment, daratumumab or the daratumumab/hyaluronidase combination is used in combination with a proteasome inhibitor and a steroid. In another embodiment, daratumumab or daratumumab/hyaluronidase combination is used in combination with a proteasome inhibitor, an alkylating agent and a steroid. In another embodiment, daratumumab or the daratumumab/hyaluronidase combination is used in combination with a proteasome inhibitor, an IMiD and a steroid. Appropriate proteasome inhibitor, alkylating agent, IMiD or steroid dosing can also be obtained from prescribing information. In some cases, the proteasome inhibitor is bortezomib. In some cases, the proteasome inhibitor is carfilzomib. In some cases, the alkylating agent is melphalan. In some cases, the steroid is prednisone. In some cases, the steroid is dexamethasone. In one embodiment, the IMiD is lenalidomide. In one embodiment, the IMiD is pomalidomide. In one embodiment, the IMiD is thalidomide.

In an example, the subject has been newly diagnosed with multiple myeloma and is ineligible for autologous stem cell transplant and the daratumumab is administered in combination with bortezomib, melphalan and prednisone (6-week cycle regimen). Further to this embodiment, the bortezomib, can be administered by subcutaneous injection at a dose of 1.3 mg/m2 body surface area twice weekly at weeks, 1, 2, 4 and 5 for the first 6-week cycle (Cycle 1; 8 doses), followed by once weekly administrations at Weeks 1, 2, 4 and 5 for eight more 6-week cycles (Cycles 2-9; 4 doses per cycle). Melphalan can be administered at 9 mg/m2, and prednisone at 60 mg/m2 orally on Days 1 to 4 of the nine 6-week cycles (Cycles 1-9). 16 mg/kg Daratumumab can be administered according to Table 4. In some cases, at least 30 mg/kg of an anti-KMA antibody (e.g., an anti-KMA antibody having the CDRs of KM) can also be administered.

In an example, the subject has been newly diagnosed with multiple myeloma and is ineligible for autologous stem cell transplant and the daratumumab/hyaluronidase combination is administered in combination with bortezomib, melphalan and prednisone (6-week cycle regimen). Further to this embodiment, the bortezomib, can be administered by subcutaneous injection at a dose of 1.3 mg/m2 body surface area twice weekly at weeks, 1, 2, 4 and 5 for the first 6-week cycle (Cycle 1; 8 doses), followed by once weekly administrations at Weeks 1, 2, 4 and 5 for eight more 6-week cycles (Cycles 2-9; 4 doses per cycle). Melphalan can be administered at 9 mg/m2, and prednisone at 60 mg/m2 orally on Days 1 to 4 of the nine 6-week cycles (Cycles 1-9). The 1800 mg daratumumab/30,000 units hyaluronidase can be administered subcutaneously according to Table 4. In some cases, at least 30 mg/kg of an anti-KMA antibody (e.g., an anti-KMA antibody having the CDRs of KM) can also be administered.

TABLE 4
Daratumumab or daratumumab/hyaluronidase combination
dosing schedule in combination with Bortezomib,
Melphalan and Prednisone ([VMP], 6-week cycle).

| Weeks | Schedule |
|---|---|
| Weeks 1 to 6 | Weekly (total of 6 doses) |
| Weeks 7 to 54^{a} | Every three weeks (total of 16 doses) |
| Week 55 onwards until | Every four weeks |
| disease progression^{b} | |

^{a}First dose of the every-3-week dosing schedule is given at week 7.
^{b}First dose of the every-4-week dosing schedule is given at week 55.

In an example, the subject has been newly diagnosed with multiple myeloma and is eligible for autologous stem cell transplant (ASCT) and the daratumumab is administered in combination with bortezomib, thalidomide and dexamethasone (4-week cycle regimen). Further to this embodiment, the bortezomib, can be administered by subcutaneous injection or intravenous (IV) injection at a dose of 1.3 mg/m2 body surface area twice weekly for two weeks (Days 1, 4, 8 and 11) of repeated 28-day (4-week) induction treatment cycles (cycles 1-4) and two consolidation cycles (Cycles 5 and 6) following ASCT after Cycle 4. Thalidomide can be administered orally at 100 mg daily during the six bortezomib cycles. Dexamethasone (oral or intravenous) can be administered at 40 mg on Days 1, 2, 8, 9, 15, 16, 22 and 23 of Cycles 1 and 2, and at 40 mg on Days 1-2 and 20 mg on subsequent dosing days (Days 8, 9, 15, 16) of Cycles 3-4. Dexamethasone 20 mg can be administered on Days 1, 2, 8, 9, 15, 16 in Cycles 5 and 6. On the days of daratumumab infusion, the dexamethasone dose can be administered intravenously as a pre-infusion medication. 16 mg/kg Daratumumab can be administered according to Table 5. In some cases, at least 30 mg/kg of an anti-KMA antibody (e.g., an anti-KMA antibody having the CDRs of KM) can also be administered

In an example, the subject has been newly diagnosed with multiple myeloma and is eligible for autologous stem cell transplant (ASCT) and the daratumumab/hyaluronidase combination is administered in combination with bortezomib, thalidomide and dexamethasone (4-week cycle regimen). Further to this embodiment, the bortezomib, can be administered by subcutaneous injection or intravenous (IV) injection at a dose of 1.3 mg/m2 body surface area twice weekly for two weeks (Days 1, 4, 8 and 11) of repeated 28-day (4-week) induction treatment cycles (cycles 1-4) and two consolidation cycles (Cycles 5 and 6) following ASCT after Cycle 4. Thalidomide can be administered orally at 100 mg daily during the six bortezomib cycles. Dexamethasone (oral or intravenous) can be administered at 40 mg on Days 1, 2, 8, 9, 15, 16, 22 and 23 of Cycles 1 and 2, and at 40 mg on Days 1-2 and 20 mg on subsequent dosing days (Days 8, 9, 15, 16) of Cycles 3-4. Dexamethasone 20 mg can be administered on Days 1, 2, 8, 9, 15, 16 in Cycles 5 and 6. On the days of daratumumab infusion, the dexamethasone dose can be administered intravenously as a pre-infusion medication. The 1800 mg daratumumab/30,000 units hyaluronidase can be administered subcutaneously according to Table 5. In some cases, at least 30 mg/kg of an anti-KMA antibody (e.g., an anti-KMA antibody having the CDRs of KM) can also be administered

TABLE 5
Daratumumab or Daratumumab/Hyaluronidase combination Dosing
Schedule in Combination with Bortezomib, Thalidomide
and Dexamethasone ([VTd]; 4-Week Cycle).

| Treatment Phase | Weeks | Schedule |
|---|---|---|
| Induction | Weeks 1 to 8 | Weekly (total of 8 doses) |
| | Weeks 9 to 16^{a} | Every two weeks (total of 4 |
| | | doses) |

Stop for high dose chemotherapy and ASCT

| | | |
|---|---|---|
| Consolidation | Weeks 1 to 8^{b} | Every two weeks (total of 4 |
| | | doses) |

^{a}First dose of the every-2-week dosing schedule is given at week 9.
^{b}First dose of the every-2-week dosing schedule is given at week 1 upon re-initiation of treatment following ASCT.

The consolidation phase of treatment can begin a minimum of 30 days post-ASCT, when the patient has recovered sufficiently, and engraftment is complete.

In one embodiment, the subject has multiple myeloma and has received at least one prior therapy and the daratumumab is administered in combination with bortezomib and dexamethasone (3-week cycle). Bortezomib can be administered by SC injection or IV injection at a dose of 1.3 mg/m2 body surface area twice weekly for two weeks (Days 1, 4, 8, and 11) of repeated 21 day (3-week) treatment cycles, for a total of 8 cycles. Dexamethasone can be administered orally at a dose of 20 mg on Days 1, 2, 4, 5, 8, 9, 11, and 12 of each of the 8 bortezomib cycles (80 mg/week for two out of three weeks of the bortezomib cycle) or a reduced dose of 20 mg/week for patients>75 years, BMI<18.5, poorly controlled diabetes mellitus or prior intolerance to steroid therapy. On the days of daratumumab infusion, 20 mg of the dexamethasone dose can be administered as a pre-infusion medication. For patients on a reduced dexamethasone dose, the entire 20 mg dose can be given as a daratumumab pre-infusion medication. Bortezomib and dexamethasone can be given for 8 three-week cycles in both treatment arms; whereas daratumumab can be given until disease progression. 16 mg/kg daratumumab is administered according to Table 6. In some cases, at least 30 mg/kg of an anti-KMA antibody (e.g., an anti-KMA antibody having the CDRs of KM) can also be administered.

In one embodiment, the subject has multiple myeloma and has received at least one prior therapy, and the daratumumab/hyaluronidase combination is administered in combination with bortezomib and dexamethasone (3-week cycle). Bortezomib can be administered by SC injection or IV injection at a dose of 1.3 mg/m2 body surface area twice weekly for two weeks (Days 1, 4, 8, and 11) of repeated 21-day (3-week) treatment cycles, for a total of 8 cycles. Dexamethasone can be administered orally at a dose of 20 mg on Days 1, 2, 4, 5, 8, 9, 11, and 12 of each of the 8 bortezomib cycles (80 mg/week for two out of three weeks of the bortezomib cycle) or a reduced dose of 20 mg/week for patients>75 years, BMI<18.5, poorly controlled diabetes mellitus or prior intolerance to steroid therapy. The 1800 mg daratumumab/30,000 units hyaluronidase can be administered subcutaneously according to Table 6. In some cases, at least 30 mg/kg of an anti-KMA antibody (e.g., an anti-KMA antibody having the CDRs of KM) can also be administered.

TABLE 6
Daratumumab or Daratumumab/Hyaluronidase combination dosing schedule
Dosing Schedule with Bortezomib and Dexamethasone (3-Week Cycle).

| Weeks | Schedule |
|---|---|
| Weeks 1 to 9 | Weekly (total of 9 doses) |
| Weeks 10 to 24^{a} | Every three weeks (total of 5 doses) |
| Week 25 onwards until disease | Every four weeks |
| progression^{b} | |

^{a}First dose of the every-3-week dosing schedule is given at week 10.
^{b}First dose of the every-4-week dosing schedule is given at week 25.

In one embodiment, the subject has multiple myeloma and has received at least one to three prior lines of therapy and the daratumumab is administered in combination with carfilzomib and dexamethasone (4-week cycle). Carfilzomib can be administered intravenously at a dose of 20 mg/m2 in Cycle 1 on Days 1 and 2; at a dose of 56 mg/m2 in Cycle 1 on Days 8, 9, 15, and 16; and at a dose 56 mg/m2 on Days 1, 2, 8, 9, 15, and 16 of each 28-day cycle thereafter. Dexamethasone 20 mg can be administered orally or intravenously on Days 1, 2, 8, 9, 15 and 16 and then 40 mg orally or intravenously on Day 22 of each 28-day cycle. Dosing of dexamethasone can otherwise be split across days when carfilzomib is administered. Treatment can be continued until disease progression or unacceptable toxicity. Daratumumab is administered according to Table 7. Daratumumab can be administered intravenously at a dose of 8 mg/kg in Cycle 1 on Days 1 and 2. Thereafter, daratumumab can be administered intravenously at a dose of 16 mg/kg on Days 8, 15 and 22 of Cycle 1; Days 1, 8 and 15 and 22 of Cycle 2; Days 1 and 15 of Cycles 3 to 6; and Day 1 of each 28-day cycle until disease progression. In some cases, at least 30 mg/kg of an anti-KMA antibody (e.g., an anti-KMA antibody having the CDRs of KM) can also be administered.

In one embodiment, the subject has multiple myeloma and has received at least one to three prior lines of therapy and the daratumumab is administered in combination with once-weekly carfilzomib and dexamethasone. Carfilzomib can be administered intravenously once weekly at a dose of 20 mg/m2 on Cycle 1 Day 1 and escalated to dose of 70 mg/m2 on Cycle 1 Days 8 and 15, and Days 1, 8, and 15 of each subsequent 28-day cycle. In Cycles 1 and 2, dexamethasone 20 mg can be administered orally or intravenously on Days 1, 2, 8, 9, 15, 16, 22 and 23; in cycles 3 to 6, dexamethasone 20 mg can be administered orally or intravenously on Days 1, 2, 15 and 16 and at a dose of 40 mg on Day 8 and 22; and in cycles 7 and thereafter, dexamethasone 20 mg can be administered orally or intravenously on Days 1 and 2 and at a dose of 40 mg on Days 8, 15, and 22. Treatment can be continued until disease progression or unacceptable toxicity. Daratumumab can be administered according to Table 7. Daratumumab can be administered intravenously at a dose of 8 mg/kg in Cycle 1 on Days 1 and 2. Thereafter, daratumumab can be administered intravenously at a dose of 16 mg/kg on Days 8, 15 and 22 of Cycle 1; Days 1, 8 and 15 and 22 of Cycle 2; Days 1 and 15 of Cycles 3 to 6; and Day 1 of each 28-day cycle until disease progression. In some cases, at least 30 mg/kg of an anti-KMA antibody (e.g., an anti-KMA antibody having the CDRs of KM) can also be administered.

TABLE 7
Daratumumab dosing schedule Dosing Schedule with Carfilzomib and
Dexamethasone (4-Week Cycle).

| Weeks | Daratumumab Dose^{c} | Schedule |
|---|---|---|
| Week 1 | 8 mg/kg | days 1 and 2 (total 2 doses) |
| Weeks 2 to 8 | 16 mg/kg | Weekly (total of 7 doses) |
| Weeks 9 to 24^{a} | 16 mg/kg | Every two weeks (total of 8 |
| | | doses) |
| Week 25 onwards until | 16 mg/kg | Every four weeks |
| disease progression^{b} | | |

^{a}First dose of the every-2-week dosing schedule is given at week 9.
^{b}First dose of the every-4-week dosing schedule is given at week 25.
^{c}Based on actual body weight.

In one embodiment, the subject has been newly diagnosed with multiple myeloma, and the daratumumab/hyaluronidase combination is administered in combination with bortezomib, lenalidomide and dexamethasone (3-week cycle). Bortezomib can be administered by SC injection at a dose of 1.3 mg/m2 body surface area twice weekly for two weeks (Days 1, 4, 8, and 11) of repeated 28-day for weeks 1-16 during induction and weeks 1-8 during consolidation. Lenalidomide can be administered orally at 25 mg daily (days 1-21) during weeks 1-16 during induction and weeks 1-8 during consolidation. Dexamethasone (oral or intravenous) can be administered at 40 mg on Days 1~4 and Days 9-12 during weeks 1-16 during induction and weeks 1-8 during consolidation. On the days of the daratumumab/hyaluronidase combination injection, the dexamethasone dose can be administered orally or intravenously as a pre-injection medication. The 1800 mg daratumumab/30,000 units hyaluronidase can be administered subcutaneously according to Table 8. In some cases, at least 30 mg/kg of an anti-KMA antibody (e.g., an anti-KMA antibody having the CDRs of KM) can also be administered.

TABLE 8
Daratumumab/Hyaluronidase combination Dosing Schedule in Combination
with Bortezomib, Lenalidomide and Dexamethasone (4-Week Cycle).

| Treatment Phase | Weeks | Schedule |
|---|---|---|
| Induction | Weeks 1 to 8 | Weekly (total of 8 doses) |
| | Weeks 9 to 16^{a} | Every two weeks (total of 4 |
| | | doses) |

Stop for high dose chemotherapy and ASCT

| | | |
|---|---|---|
| Consolidation | Weeks 1 to 8^{b} | Every two weeks (total of 4 |
| | | doses) |

^{a}First dose of the every-2-week dosing schedule is given at week 9.
^{b}First dose of the every-2-week dosing schedule is given at week 1 upon re-initiation of treatment following ASCT.

In an example, pre-infusion medication can be administered 1 hour to 3 hours before every daratumumab infusion or daratumumab/hyaluronidase combination injection:

In some cases, methylprednisolone 100 mg (or equivalent) can be administered intravenously or orally as a monotherapy. In some cases, following a second infusion of daratumumab or second injection of the daratumumab/hyaluronidase combination, methylprednisolone can be administered at a dose of 60 mg (or equivalent) either orally or intravenously.

In some cases, dexamethasone 20 mg (or equivalent) can be administered orally or intravenously prior to every daratumumab infusion or daratumumab/hyaluronidase combination injection. When dexamethasone is the background regimen-specific corticosteroid, the dexamethasone dose that is part of the background regimen can serve as pre-medication on daratumumab infusion or daratumumab/hyaluronidase combination injection days. In some cases, background regimen-specific corticosteroids (e.g. prednisone) are not administered on daratumumab infusion or daratumumab/hyaluronidase combination injection days when patients have received dexamethasone (or equivalent) as a pre-medication.

In some cases, acetaminophen 650 mg to 1,000 mg is administered orally.

In some cases, diphenhydramine 25 mg to 50 mg (or equivalent) is administered orally or intravenously.

In an example, post-infusion medication can be administered.

In some cases, methylprednisolone 20 mg (or an equivalent dose of an intermediate- or long-acting corticosteroid) orally for 2 days starting the day after the administration of daratumumab or the daratumumab/hyaluronidase combination.

In some cases, oral methylprednisolone is administered at a dose of less than or equal to 20 mg (or an equivalent dose of an intermediate- or long-acting corticosteroid) beginning the day after the administration of a daratumumab infusion or daratumumab/hyaluronidase combination injection.

If a background regimen-specific corticosteroid (e.g. dexamethasone, prednisone) is administered the day after the daratumumab infusion or the daratumumab/hyaluronidase combination injection, additional corticosteroids may not be needed.

For patients with a history of chronic obstructive pulmonary disease, short and long-acting bronchodilators and inhaled corticosteroids can be prescribed. Following the first 4 daratumumab infusions or 3 doses of the daratumumab/hyaluronidase combination injection, these additional post-infusion medications may be discontinued, if the patient does not experience a major infusion-related reaction.

### Kits

The combination dosing regimen according to the present disclosure can be provided in a kit or pack. For example, the therapeutic combination disclosed herein may be packaged in a suitable container with written instructions for treating an above referenced condition (e.g., multiple myeloma).

In an example, an anti-KMA antibody may be provided in a single dose or multiple doses. For example, the anti-KMA antibody may be provided in a single dose container such as a vial. In an example, the anti-KMA antibody may be provided in multiple single dose containers sufficient for at least 8 weeks of treatment. For example, the kit or pack may comprise at least 8 vials of pre-prepared KM.

In an example, an IMiD or a CELMOD may be provided in a single dose or multiple doses. For example, the IMiD or the CELMOD may be provided in a single dose container such as a pre-packaged pill. In an example, the IMiD or the CELMOD may be provided in multiple single dose containers sufficient for at least 8 weeks of treatment. For example, the kit or pack may comprise at least 60 pre-packaged pills of lenalidomide. In another example, the kit or pack may comprise at least 60 pre-packaged pills of pomalidomide.

In an example, a steroid may be provided in a single dose or multiple doses. For example, the steroid may be provided in a single dose container such as a pre-packaged pill. In an example, the steroid may be provided in multiple single dose containers sufficient for at least 8 weeks of treatment. For example, the kit or pack may comprise at least 8 pre-packaged pills of dexamethasone.

In an example, daratumumab may be provided in a single dose or multiple doses. For example, daratumumab may be provided as a single dose solution such as in a single-dose vial. In an example, daratumumab may be provided in multiple single dose solutions sufficient for at least 8 weeks of treatment. For example, the kit or pack may comprise at least 8 pre-packaged vials of daratumumab. Each single-dose vial can comprise 100 mg/5 ml (20 mg/ml) or 400 mg/20 ml (20 mg/ml) of daratumumab.

In an example, daratumumab may be provided in a single dose or multiple doses. For example, daratumumab may be provided as a single dose solution such as in a single-dose vial. In an example, daratumumab may be provided in multiple single dose solutions sufficient for at least 8 weeks of treatment. For example, the kit or pack may comprise at least 8 pre-packaged vials of daratumumab. Each single-dose vial can comprise 100 mg/5 ml (20 mg/ml) or 400 mg/20 ml (20 mg/ml) of daratumumab.

In an example, daratumumab/hyaluronidase combination may be provided in a single dose or multiple doses. For example, daratumumab/hyaluronidase combination may be provided as a single dose solution such as in a single-dose vial. In an example, daratumumab/hyaluronidase combination may be provided in multiple single dose solutions sufficient for at least 8 weeks of treatment. For example, the kit or pack may comprise at least 8 pre-packaged vials of daratumumab/hyaluronidase combination. Each single-dose vial can comprise a 1,800 mg daratumumab and 30,000 units hyaluronidase per 15 mL (120 mg and 2,000 units/mL) solution in a single-dose vial.

In an example, a proteasome inhibitor may be provided in a single dose or multiple doses. For example, the proteasome inhibitor may be provided in a single dose container such as a pre-packaged pill. In an example, the proteasome inhibitor may be provided in multiple single dose solutions sufficient for at least 8 weeks of treatment. For example, the kit or pack may comprise at least 8 pre-packaged vials of proteasome inhibitor (e.g., bortezomib or carfilzomib).

In an example, an alkylating agent may be provided in a single dose or multiple doses. For example, the alkylating agent may be provided in a single dose container such as a pre-packaged pill. In an example, the alkylating agent may be provided in multiple single dose containers sufficient for at least 8 weeks of treatment. For example, the kit or pack may comprise at least 8 pre-packaged pills of alkylating agent (e.g., melphalan).

The present disclosure includes the following non-limiting Examples.

### EXAMPLES

### Example 1—Materials and Methods

### Early Investigational Plan

An early investigational study looking at four dose levels of KM (0.3 mg/kg, 1.0 mg/kg, 3.0 mg/kg, 10 mg/kg) was undertaken. The assay data was taken up to Day 45 for patients in these dose groups, and showed that KM could still be detected until at least Day 15, even at the lower doses. This early data suggested that anti-KMA antibodies were not subject to the phenomenon known as antigen sink, and as a result there was a higher probability that the anti-KMA antibody would be exposed to KMA cells without the need for higher dosage levels. As such, taken together with the known potential for monoclonal antibody induced immunotoxicity at higher doses, anti-KMA antibodies were considered good candidates for low dosing. Accordingly, the phase IIb study described below was designed based on these low doses.

### Study Design

Phase IIb, multi-centre, open label sequential cohort study evaluating kappamab (KM) alone (KM, Stage 1) and then KM in combination with lenalidomide (LEN) and dexamethasone (DEX) (KM-Rd, Stage 2) in relapsed/refractory multiple myeloma (KRRMM). Key inclusion criteria for both stages were progressive kappa-restricted multiple myeloma (KMM) (as per International Myeloma Working Group (IMWG) criteria, Kumar et al. 2016), 1-3 prior lines of therapy and no prior LEN exposure.

### Study Stages, Dosing and Stopping Criteria

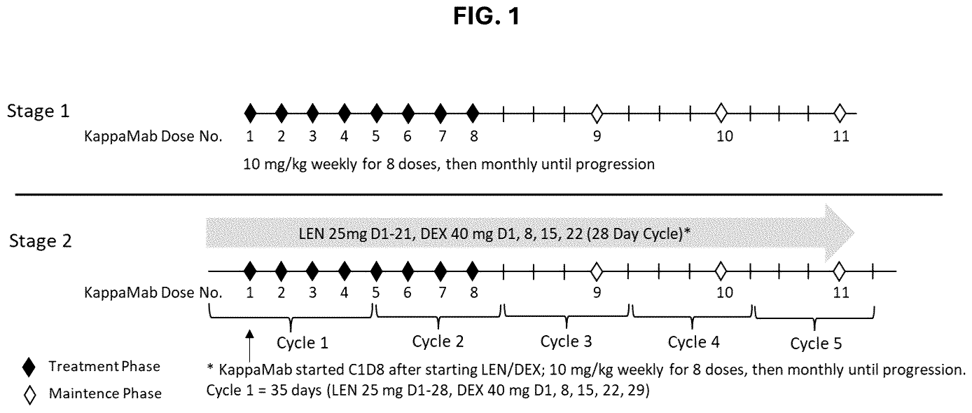

Recruitment was planned for 60 patients in total, with an initial intention to treat 30 patients per stage. In Stage 1, patients received KM (10 mg/kg intravenous (IV) infusion) weekly for 8 weeks then every 4 weeks as maintenance. In Stage 2, KM dosing was as per Stage 1 with the addition of LEN, 25 mg Days 1-21 of each 28-day cycle and DEX 40 mg weekly, apart from Cycle 1 which was of 35 days duration (28-day cycles from Cycle 2 onwards) as patients commenced LEN for 21 days and DEX 1 week prior to starting KM (FIG. 1). All patients received anti-viral, thromboembolic prophylaxis and osteolytic prophylaxis as per institutional practice. Treatment continued until unacceptable toxicity, progression, death or withdrawal of consent. Response rates and any adverse events (AEs) were periodically assessed.

### Combination KM-Rd Cohort Compared to Matched Controls (Rd-MRDR)

The overall response rate (ORR), overall survival (OS) and progression free survival (PFS) of the KM-Rd cohort was compared to a contemporaneous control group of KMM patients who had received Rd for RRMM (Rd-MRDR group), identified via the Australian and New Zealand Myeloma and Related Diseases Registry (MRDR) (www dot mrdr dot net dot au/), who were matched for age, gender and prior lines of therapy (Table 10). The patients also had a similar mix of types of prior therapies, number of prior lines and cytogenetic risk. The KM-Rd group had a higher percentage of patients with baseline ISS stage 2 diagnoses (55%) versus the MRDR cohort (45%) and the KM cohort (22%). Both the KM-Rd and MRDR cohorts had 26% of patients with ISS stage 3 at baseline, while the KM cohort had 33% who were ISS stage 3 at baseline. A limitation of this Rd-MRDR dataset was that patients had their disease status recorded only every 4 months, however an advantage of this dataset was that OS was regularly cross referenced with the Australian Institutes of Health and Welfare National Death Index (www dot aihw dot gov dot au/about-our-data/our-data-collections/national-death-index). Importantly, both the KM-Rd cohort and the Rd MRDR controls were treated within the same time period and with similar supportive care and access to both reimbursed pomalidomide and carfilzomib but neither CD38 nor BCMA targeting immune therapies.

### Study Endpoints

The primary endpoint for both Stage 1 and Stage 2 was the clinical benefit rate (CBR). The primary and secondary endpoints are summarised in Table 9. Patients were evaluated every 28 days for both OS and PFS while on study. Comparison of CBR, ORR, OS and PFS between KM-Rd and Rd-MRDR groups was done after the data cut-off. Assessment of duration of response (DoR) and time to next treatment (TTNT) was done in the KM-Rd group only; DoR and TTNT data were not available for the Rd-MRDR group.

TABLE 9
Study endpoints and definitions

| Primary Endpoint | Definition |
|---|---|
| Clinical Benefit | Minimal response (MR) or better as per the IMWG uniform response |
| Rate (CBR) | criteria¹¹ |

Secondary Endpoints

| | |
|---|---|
| Overall response rate | The proportion of patients who achieved a ≥ partial response [PR] as per |
| (ORR) | the IMWG uniform response criteria¹¹ |

Safety Endpoints:

| | |
|---|---|
| Overall survival (OS) | Measured from the date of first dose of study drug until the date of death |
| | from any cause |
| Progression free survival | Measured from the date of first dose of study drug until the earliest of |
| (PFS) | the date of relapse, progression or death from any cause |
| Infusion-related Reactions | Symptoms of an IRR can include fever, chills, shakes, itching, rash, |
| (IRRs) | hyper-or hypotension, difficulty breathing, vomiting or headache. |
| Adverse Events (AEs) | Unintended changes in signs or symptoms of the body; incidence and |
| | severity of AEs were assessed using the CTCAE version 4.0. |
| Duration of response | Restricted to patients who achieved PR, measured from the date on |
| (DoR) | which a PR or better was first observed; patients who were not known to |
| | have progressed or died were censored at the date of their last evaluation |
| Time to next treatment | Measured from the date of first dose of study drug until the date that next |
| (TTNT) | therapy commenced or the date of death if another therapy did not |
| | commence, and, censored at the date of last contact |

### Statistical Approach

Demographic and baseline characteristics were compared between the KM-Rd cohort and the Rd-MRDR controls using a Chi-squared test. CBR and ORR were estimated as simple percentages and 95% credible intervals (CI) were calculated as specified in the protocol using the pbeta and qbeta functions in R Version 4. A descriptive analysis of each of the time-to-event endpoints (DoR, TTNT, OS and PFS) in each stage used the Kaplan-Meier (product-limit) method to estimate the survival functions, with conventional 95% confidence intervals (CI) for the median survival time, DoR, TTNT, OS and PFS calculated using the Brookmeyer and Crowley method. Median potential follow-up was estimated by reversing the censor indicators in the Kaplan-Meier analyses. TTNT and the competing risk of death before a switch to another therapy were investigated by the calculation of cumulative incidence functions using the cmprsk library (Version 2.2-11) in R Version 4. The comparisons of the time-to-event endpoints comparing the KM cohort with KM-Rd cohort, and the KM-Rd cohort with the Rd-MRDR controls were undertaken using log-rank tests and were summarised as hazard ratios with 95% confidence intervals calculated using Cox proportional hazards regression models. These analyses were performed using SPSS v27 and Stata 16.1. A two-tailed p-value<0.05 was taken to indicate statistical significance.

### Example 2—Clinical Assessment

### Kappamab Dose Cohorts

Fifty-nine of the planned 60 patients were enrolled between November 2016 and July 2019. Demographics and baseline characteristics are described for Stage 1, Stage 2 and the Rd-MRDR patients in Table 10. Recruitment to Stage 1 (KM cohort) was terminated early (n=19), as the first PoC criterion was not met and posterior predictive probability calculations for CBR indicated that PoC was unlikely to be declared. Recruitment to Stage 2 (KM-Rd cohort) was expanded from 30 to 40 patients.

TABLE 10
Demographics and Baseline Characteristics

| | Stage 1 | Stage 2 | MRDR | p- |
|---|---|---|---|---|
| Factor | KM | KM-Rd | Rd Controls | value¹ |
| N | 19 | 40 | 77 | |
| Age, years (IQR) | 60 (40-82) | 68 (63-74) | 69 (61-74) | NS |
| Time from diagnosis, | | 34 (18-47) | 26 (17-38) | NS |
| months (IQR) | | | | |
| Gender | | | | NS |
| Male | 58% | 55% | 55% | |
| Female | 42% | 45% | 45% | |
| ISS at diagnosis | | | | NS |
| 1 | 44% | 19% | 29% | |
| 2 | 22% | 55% | 45% | |
| 3 | 33% | 26% | 26% | |
| Paraprotein type | | | | <0.001 |
| IgG | 74% | 50% | 70% | |
| IgA | 5% | 25% | 29% | |
| IgD | 0% | 0% | 1% | |
| Light chain only | 21% | 25% | 0% | |
| Prior lines of treatment | | | | NS |
| 1 | 42% | 55% | 39% | |
| 2 | 47% | 35% | 44% | |
| 3 | 11% | 0% | 2% | |
| 4 | 0% | 0% | 4% | |
| 5 | 0% | 0% | 1% | |
| Prior therapy | | | | NS |
| PI | 100% | 93% | 97% | |
| Refractory to PI | 5% | 18% | 21% | |
| Thalidomide | 47% | 55% | 50% | |
| ASCT | 53% | 53% | 47% | |
| Cytogenetic risk² | | | | NS |
| Standard | 89% | 77% | 83% | |
| High | 11% | 23% | 18% | |

¹Stage 2 versus Rd-MRDR controls;
²High risk = t(4;14), del17p or t(14;16);
IQR = interquartile range

At the study censor date, two patients in Stage 2 (5%) remained on. In the other patients, reasons for discontinuation were progression (Stage 1=16/19 [84%], Stage 2=31/40 [70%]), withdrawal of consent unrelated to KM toxicity (Stage 1=3/19 [16%], Stage 2=4/40 [10%]), and toxicity due to LEN administration (Stage 2=3/40 [7.5%]). In Stage 2, two patients (5%) died on study from causes that were considered by the investigator to be not related to study drug (pneumonia, n=1; unknown cause, n=1).

### Primary and Secondary Endpoints for Stage 1 and Stage 2

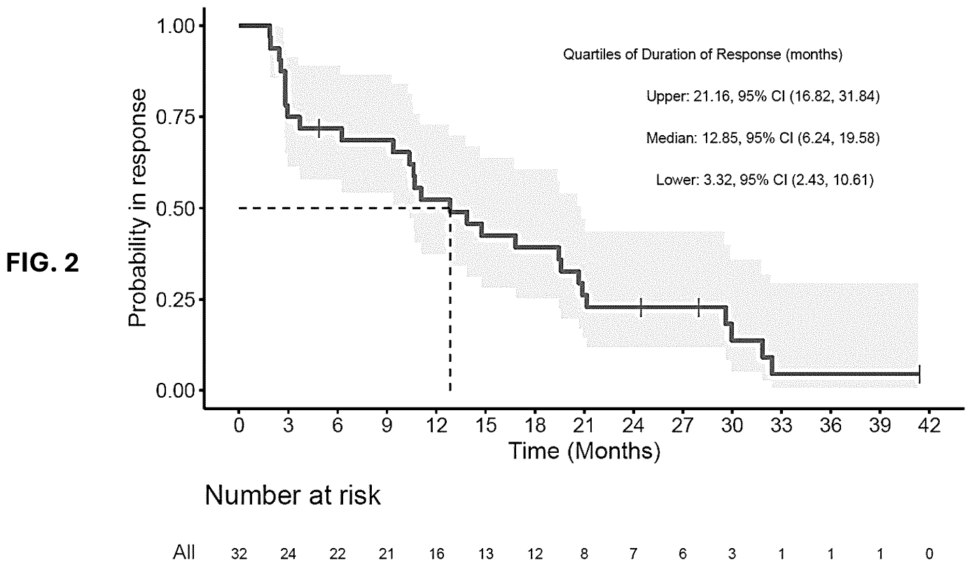

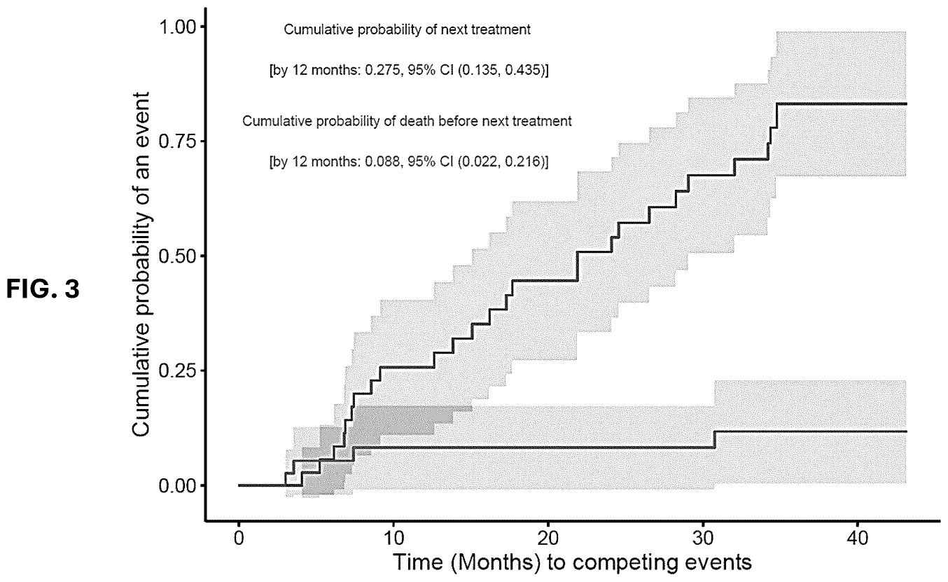

The CBR for Stage 1 and Stage 2 were 5% (95% CI: 0.5-21.1%) [1/19, PR=1] and 93% (95% CI: 79.9-97.3%) [37/40; CR=3/37 (8.1%), VGPR=11/37 (29.7%), PR=19/37 (51.4%), MR=4/37 (10.8%), respectively, with ORR 5% [1/19] and 83% (95% CI: 67.7-91.1%) [33/40], respectively. The median PFS for Stage 1 and Stage 2 was 2.0 months (95% CI: 0.0-4.7 months) and 12.7 months (95% CI 6.6 m to 18.8 months) (HR 0.25, 95% CI 0.13 to 0.47, p<0.001), respectively, with median OS not reached for both stages. Median DoR and TTNT in Stage 2 were 12.9 months (95% CI 6.2 to 19.6 months) (FIG. 2) and 21.9 months (95% CI 12.6-28.3 months), respectively. Moreover, for Stage 2 at 12 and 24 months the cumulative incidences of switching to another treatment were 27.5% (95% CI 13.5 to 43.5%) and 55.1% (35.3 to 71.1%) respectively and the cumulative incidence of death before a switch to another treatment remained at 8.8% (95% CI 2.2-21.6%) from 7.4 to 30.7 months (FIG. 3).

### Comparison of the Stage 2 KM-Rd and Rd-MRDR Cohorts

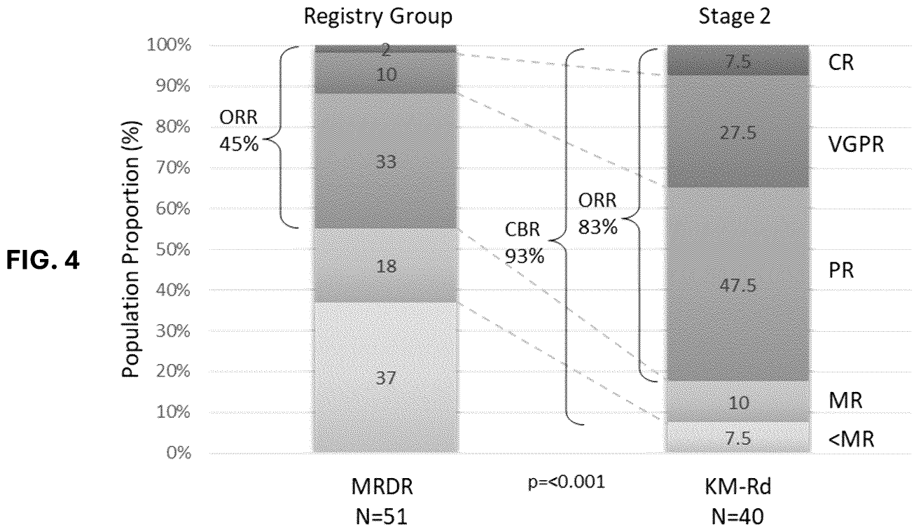

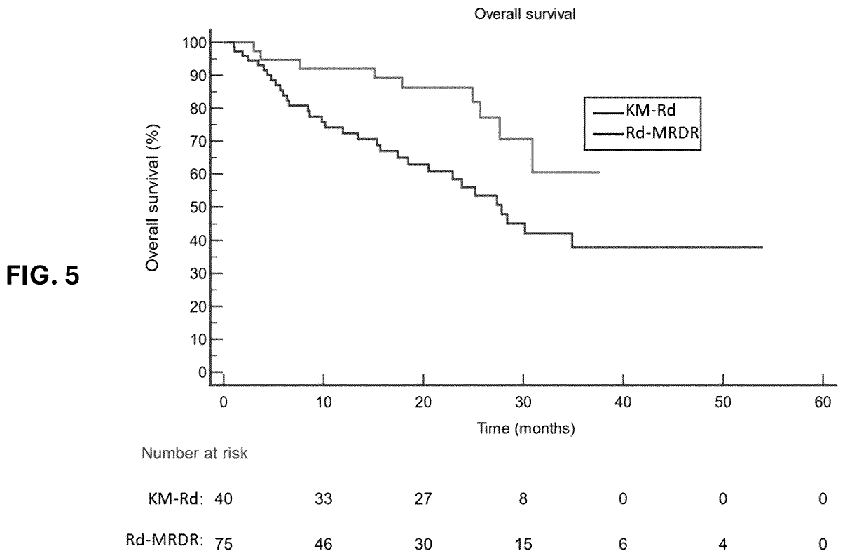

Fifty-one of 77 patients (66%) in the Rd-MRDR cohort had response data available for both CBR and ORR (Table 11). KM-Rd was superior to Rd-MRDR-93% versus 63% for CBR and 83% versus 45% for ORR respectively, both p<0.001 (FIG. 4). Seventy-five of 77 patients in the Rd-MRDR control group had data for survival with no difference in PFS between KM-Rd and Rd-MRDR-median PFS 12.7 months versus 10.3 months, 95% CI 6.23 to 23.57, p=0.55 but with a significant OS advantage-median OS not reached vs 27.8 months, p=0.02, HR 0.46 (95% CI 0.25 to 0.87) (FIG. 5).

TABLE 11
Responses, CBR and ORR-Rd-MRDR Controls versus Stage 2 KM-Rd

| | | | | |
|---|---|---|---|---|
| Group | N | % | p-value | Odds Ratio (95% CI) |

Clinical Benefit Rate

| | | | | |
|---|---|---|---|---|
| Rd-MRDR n = 51 | 32 | 63 | <0.001 | 7.3 (2.0-27.0) |
| Stage 2 (KM-Rd) n = 40 | 37 | 93 | | |
| Overall Response Rate | | | | |
| Rd-MRDR n = 51 | 23 | 45 | <0.001 | 5.7 (2.1-15.4) |
| Stage 2 (KM-Rd) n = 40 | 33 | 83 | | |

M Protein and Serum Free Light Chain (sFLC) Data Observations in Stage 1 and 2 Patients

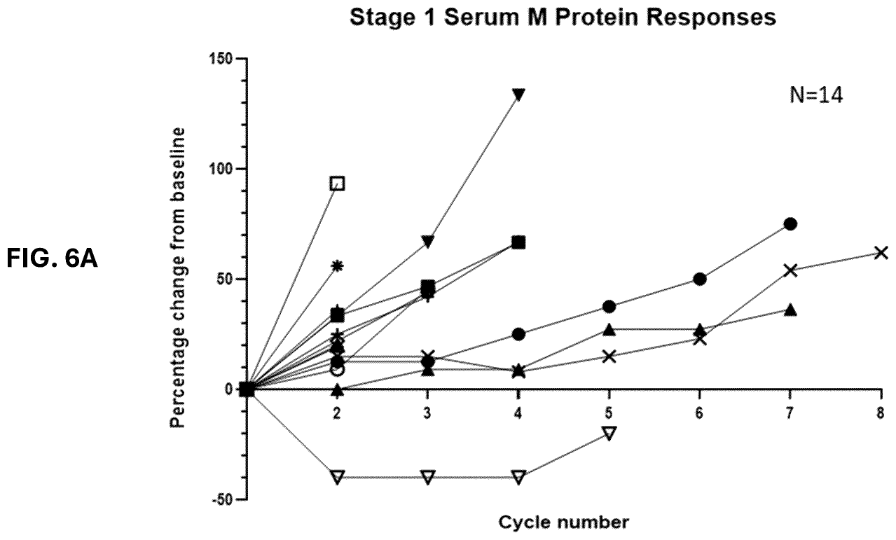

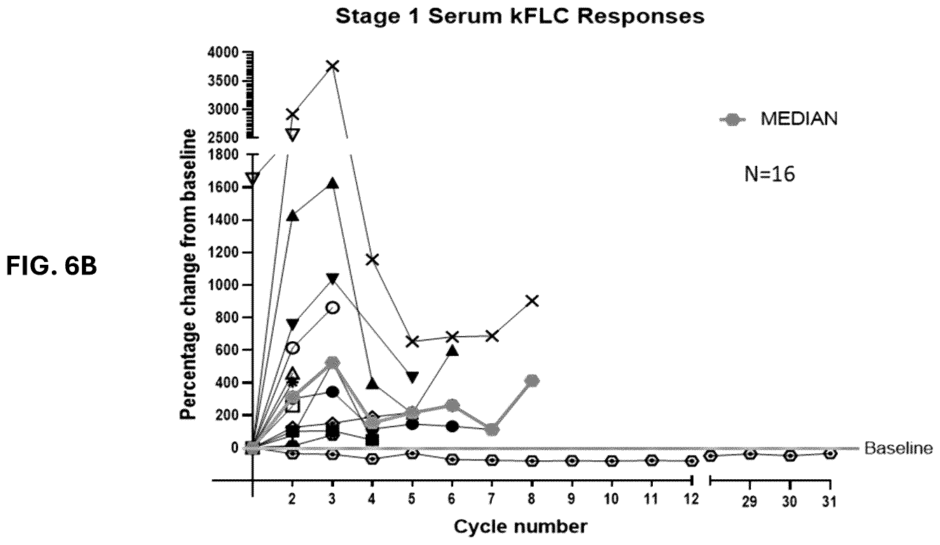

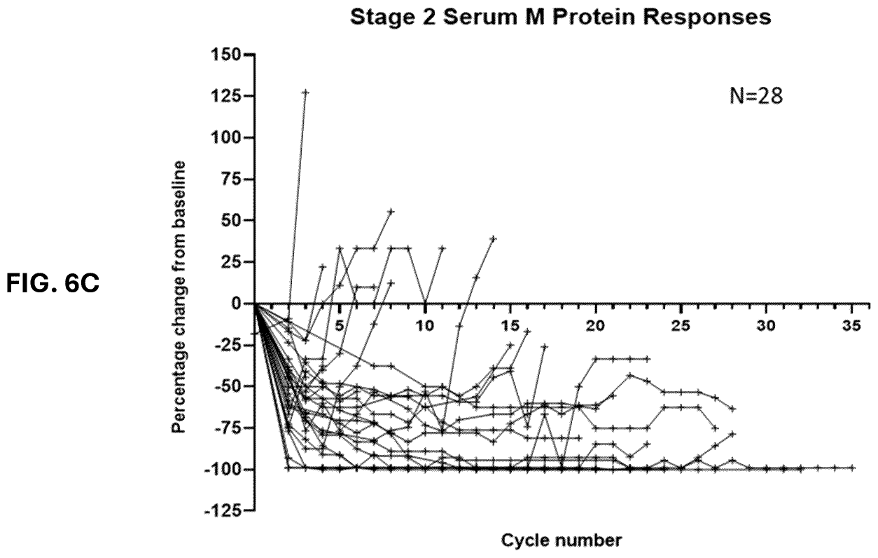

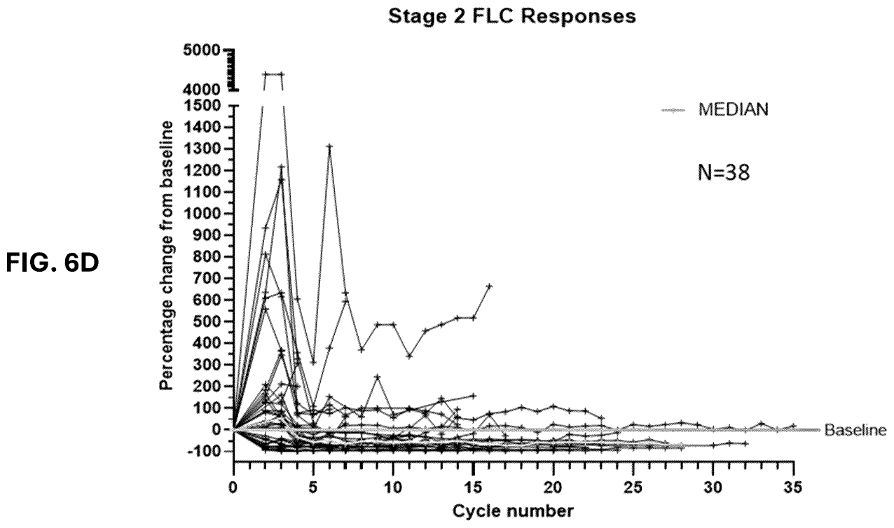

FIGS. 6A-6D shows serum M protein and kappa free light chain (KFLC) data, for each Stage. Of note in Stage 1, a patient showed a disease response based on reduced M protein levels however, their sFLC increased. In addition, one FLC-only MM patient maintained decreased sFLC levels for 31 cycles of treatment. In Stage 2, the majority of patients responded to KM-Rd (FIG. 6C). The median percent change from baseline for sFLC was elevated to Cycle 3 (i.e., following 9 doses), then it reduced and remained below baseline.

### Safety

Surprisingly, KM demonstrated a highly favourable toxicity profile even when administered as combination therapy (KM-Rd). In Stage 1, 3/19 patients (15.8%) experienced an infusion related reaction (IRR), with one Grade 1 and two Grade 2 reactions. In Stage 2, 8 IRRs, 6 with the first infusion, were observed. There was 1 Grade 3 IRR and 7 Grade 12 IRRs and no patients discontinued treatment because of IRRs. In particular, the patient with the Grade 3 IRR recovered following hydrocortisone, salbutamol and loratadine administration in the clinic, prior to hospital admission for observation. There were no haematologic toxicities reported with KM, whereas the rates of anaemia (12.5%), neutropenia (32.5%) and thrombocytopenia (18%) seen in Stage 2 with KM-Rd were as expected with Rd administration (Table 12). The most frequently reported non-haematologic AEs were fatigue, insomnia, musculoskeletal pain, peripheral neuropathy and diarrhoea (Table 13).

TABLE 12
Stage 2 Haematologic AEs (N = 40)

| | Grade-N (%) | | | | |
|---|---|---|---|---|---|
| | 1 | 2 | 3 | 4 | Total-N (%) |
| Neutropenia | 2 (5%) | 3 (7.5%) | 6 (15%) | 2 (5%) | 13 (32.5%) |
| Anaemia | 1 (2.5%) | 1 (2.5%) | 3 (7.5%) | 0 (0%) | 5 (12.5%) |
| Thrombocytopenia | 1 (2.5%) | 1 (2.5%) | 2 (5%) | 3 (7.5%) | 7 (18%) |

TABLE 13
Treatment Emergent Non-Haematologic AEs (≥10% in Stage 2)

| | Stage 1 | | Stage 2 | |
|---|---|---|---|---|
| | All grade, | Grade 3, | All Grade, | Grade 3, |
| Adverse Event | % | % | % | % |
| Rash | 16 | — | 10 | — |
| Hyperglycaemia | — | — | 10 | — |
| Fatigue | 32 | — | 30 | 3 |
| Abdominal pain | 5 | — | 10 | 3 |
| Constipation | — | — | 10 | — |
| Diarrhoea | 5 | — | 13 | — |
| Nausea | — | — | 10 | — |
| Hypophosphatemia | — | — | 10 | — |
| Infusion related | 16 | — | 13 | 3 |
| reaction | | | | |
| Peripheral | — | — | 15 | 3 |
| Neuropathy | | | | |
| Insomnia | — | — | 23 | 6 |
| Cramps | — | — | 10 | — |
| Pain | 5 | — | 20 | — |
| URTI | 5 | — | 20 | — |
| Pneumonia | — | — | 8 | 8 |
| Cellulitis | 10 | 5 | 10 | — |

The low incidence of KM-treatment related adverse events observed in clinical trials to date support doses of KM higher than 10 mg/kg in combination with immunomodulatory drug (IMiD)/cereblon E3 ligase modulator (CELMOD) and steroid therapy. Essentially, the KM-Rd safety profile recapitulated that seen with Rd (Dimopoulos et al. 2007; Weber et al. 2007) and with only infrequent (14%) and low-grade KM-related IRRs. The lack of increase in neutropenia and infection rates in the KM-Rd cohort relative to Rd administration in the literature likely reflects the highly restricted pattern of KMA expression, with KMA being found only on malignant plasma cells and limited numbers of tonsillar B-cells (Walker et al. 1985; Hutchinson et al. 2014).

### REFERENCES

Palumbo A, Chanan-Khan A, Weisel K, Nooka A K, Masszi T, Beksac M, et al. Daratumumab, Bortezomib, and Dexamethasone for Multiple Myeloma. N Engl J Med. 2016; 375(8): 754-66.
Dimopoulos M A, Oriol A, Nahi H, San-Miguel J, Bahlis N J, Usmani S Z, et al. Daratumumab, lenalidomide, and dexamethasone for multiple myeloma. New Engl J Med. 2016; 375(14): 1319-31.
Lonial S, Dimopoulos M, Palumbo A, White D, Grosicki S, Spicka I, et al. Elotuzumab therapy for relapsed or refractory multiple myeloma. N Engl J Med. 2015; 373(7): 621-31.
Attal M, Richardson P G, Rajkumar S V, San-Miguel J, Beksac M, Spicka I, et al. Isatuximab plus pomalidomide and low-dose dexamethasone versus pomalidomide and low-dose dexamethasone in patients with relapsed and refractory multiple myeloma (ICARIA-MM): a randomised, multicentre, open-label, phase 3 study. Lancet. 2019; 394(10214): 2096-107.
Walker K Z, Boux H A, Hayden G E, Goodnow C C, Raison R L. A monoclonal antibody with selectivity for human kappa myeloma and lymphoma cells which has potential as a therapeutic agent. Adv Exp Med Biol. 1985; 186:833-41.
Hutchinson A T, Jones D R, McCauley Winter P, Tangye S G, Raison R L. Cell membrane associated free kappa light chains are found on a subset of tonsil and in vitro-derived plasmablasts. Hum Immunol. 2014; 75(9): 986-90.
Asvadi P, Cuddihy A, Dunn R D, Jiang V, Wong M X, Jones D R, et al. MDX-1097 induces antibody-dependent cellular cytotoxicity against kappa multiple myeloma cells and its activity is augmented by lenalidomide. Br J Haematol. 2015; 169(3): 333-43.
Hutchinson A T, Ramsland P A, Jones D R, Agostino M, Lund M E, Jennings C V, et al. Free Ig light chains interact with sphingomyelin and are found on the surface of myeloma plasma cells in an aggregated form. J Immunol. 2010; 185(7): 4179-88.
Dunn R, Spencer A, Augustson B, Mollee P, Copeman M, Asvadi P. Phase 2a, open-label, multi-dose study of anti-kappa monoclonal antibody, MDX-1097, in relapsed kappa-chain restricted multiple myeloma with stable measurable disease. Haematol Lat. 2013; 98(s1): 776.
Kumar S, Paiva B, Anderson K C, Durie B, Landgren O, Moreau P, et al. International Myeloma Working Group consensus criteria for response and minimal residual disease assessment in multiple myeloma. The Lancet Oncology. 2016; 17(8): e328-e46.
Zonder J A, Mohrbacher A F, Singhal S, van Rhee F, Bensinger W I, Ding H, et al. A phase 1, multicenter, open-label, dose escalation study of elotuzumab in patients with advanced multiple myeloma. Blood. 2012; 120(3): 552-9.
Lokhorst H M, Plesner T, Laubach J P, Nahi H, Gimsing P, Hansson M, et al. Targeting CD38 with daratumumab monotherapy in multiple myeloma. N Engl J Med. 2015; 373(13): 1207-19.
Facon T, Kumar S, Plesner T, Orlowski R Z, Moreau P, Bahlis N, et al. Daratumumab plus Lenalidomide and Dexamethasone for Untreated Myeloma. N Engl J Med. 2019; 380(22): 2104-15.
Mateos M V, Cavo M, Blade J, Dimopoulos M A, Suzuki K, Jakubowiak A, et al. Overall survival with daratumumab, bortezomib, melphalan, and prednisone in newly diagnosed multiple myeloma (ALCYONE): a randomised, open-label, phase 3 trial. Lancet. 2020; 395(10218): 132-41.
Offidani M, Corvatta L, More S, Nappi D, Martinelli G, Olivieri A, et al. Daratumumab for the Management of Newly Diagnosed and Relapsed/Refractory Multiple Myeloma: Current and Emerging Treatments. Front Oncol. 2020; 10:624661.
Dimopoulos M, Spencer A, Attal M, Prince H M, Harousseau J L, Dmoszynska A, et al. Lenalidomide plus dexamethasone for relapsed or refractory multiple myeloma. N Engl J Med. 2007; 357(21): 2123-
Weber D M, Chen C, Niesvizky R, Wang M, Belch A, Stadtmauer E A, et al. Lenalidomide plus dexamethasone for relapsed multiple myeloma in North America. N Engl J Med. 2007; 357(21): 2133-
Bahlis N J, Dimopoulos M A, White D J, Benboubker L, Cook G, Leiba M, et al. Daratumumab plus lenalidomide and dexamethasone in relapsed/refractory multiple myeloma: extended follow-up of POLLUX, a randomized, open-label, phase 3 study. Leukemia. 2020; 34(7): 1875-84.
Mateos M V, Sonneveld P, Hungria V, Nooka A K, Estell J A, Barreto W, et al. Daratumumab, Bortezomib, and Dexamethasone Versus Bortezomib and Dexamethasone in Patients With Previously Treated Multiple Myeloma: Three-year Follow-up of CASTOR. Clin Lymphoma Myeloma Leuk. 2020; 20(8): 509-18.
Rasche L, Wasch R, Munder M, Goldschmidt H, Raab M S. Novel immunotherapies in multiple myeloma-chances and challenges. Haematologica. 2021; 106(10): 2555-65.
Shah N, Chari A, Scott E, Mezzi K, Usmani S Z. B-cell maturation antigen (BCMA) in multiple myeloma: rationale for targeting and current therapeutic approaches. Leukemia. 2020; 34(4): 985-1005.
Lonial S, Lee H C, Badros A, Trudel S, Nooka A K, Chari A, et al. Belantamab mafodotin for relapsed or refractory multiple myeloma (DREAMM-2): a two-arm, randomised, open-label, phase 2 study. Lancet Oncol. 2020; 21(2): 207-21.
Farooq A V, Degli Esposti S, Popat R, Thulasi P, Lonial S, Nooka A K, et al. Corneal Epithelial Findings in Patients with Multiple Myeloma Treated with Antibody-Drug Conjugate Belantamab Mafodotin in the Pivotal, Randomized, DREAMM-2 Study. Ophthalmol Ther. 2020; 9(4): 889-911.
Munshi N C, Anderson L D, Jr., Shah N, Madduri D, Berdeja J, Lonial S, et al. Idecabtagene Vicleucel in Relapsed and Refractory Multiple Myeloma. N Engl J Med. 2021; 384(8): 705-16.
Berdeja J G, Madduri D, Usmani S Z, Jakubowiak A, Agha M, Cohen A D, et al. Ciltacabtagene autoleucel, a B-cell maturation antigen-directed chimeric antigen receptor T-cell therapy in patients with relapsed or refractory multiple myeloma (CARTITUDE-1): a phase 1b/2 open-label study. Lancet. 2021; 398(10297): 314-24.
Van Oekelen O, Aleman A, Upadhyaya B, Schnakenberg S, Madduri D, Gavane S, et al. Neurocognitive and hypokinetic movement disorder with features of parkinsonism after BCMA-targeting CAR-T cell therapy. Nat Med. 2021; 27(12): 2099-103.

### NUMBERED EMBODIMENTS OF THE DISCLOSURE

Other subject matter contemplated by the present disclosure is set out in the following numbered embodiments:
1. A method of treating multiple myeloma in a subject in need thereof, the method comprising administering to the subject a treatment which comprises: (i) at least 30 mg/kg of an anti-KMA antibody; (ii) an immunomodulatory drug (IMiD) or cereblon E3 ligase modulator (CELMoD); and (iii) a steroid.
2. The method according to embodiment 1, wherein the anti-KMA antibody comprises a VH and a VL, the VH comprising a complementarity determining region (CDR) 1 comprising an amino acid sequence as shown in SEQ ID NO: 6, a CDR2 comprising an amino acid sequence as shown in SEQ ID NO: 7 and a CDR3 comprising a sequence as shown in SEQ ID NO: 8 and the VL comprising a CDR 1 comprising an amino acid sequence as shown in SEQ ID NO: 9, a CDR2 comprising an amino acid sequence as shown in SEQ ID NO: 10 and a CDR3 comprising a sequence as shown in SEQ ID NO: 11.
3. The method according to embodiment 1 or 2, wherein the anti-KMA antibody has a heavy chain variable region (VH) comprising a sequence set forth in SEQ ID NO: 1 and a light chain variable region (VL) comprising a sequence set forth in SEQ ID NO: 2.
4. The method according to any one of embodiments 1 to 3, wherein the IMiD is lenalidomide, pomalidomide or thalidomide.
5. The method according to any one of embodiments 1 to 4, wherein the IMiD is pomalidomide.
6. The method according to any one of embodiments 1 to 3, wherein the CELMOD is iberdomide or CC-92480.
7. The method according to any one of embodiments 1 to 6, wherein about 25 mg of the IMiD or the CELMOD is administered to the subject.
8. The method according to any one of embodiments 1 to 7, wherein the steroid is dexamethasone or prednisone.
9. The method according to any one of embodiments 1 to 8, wherein the steroid is dexamethasone.
10. The method according to any one of embodiments 1 to 9, wherein about 40 mg of the steroid is administered to the subject.
11. The method according to any one of embodiments 1 to 10, wherein treatment comprises administering to the subject in a treatment phase: (i) at least 30 mg/kg of the anti-KMA antibody once per week; (ii) the IMiD or the CELMOD once per day; and (iii) the steroid once per week.
12. The method according to embodiment 11, wherein the duration of the treatment phase is between at least 6 weeks and at least 10 weeks.
13. The method according to embodiment 11 or 12, further comprising administering to the subject the IMiD or the CELMOD once per day for at least 3 weeks prior to the start of the treatment phase.
14. The method according to any one of embodiments 11 to 13, further comprising administering a steroid to the subject at least 1 week prior to the start of the treatment phase.
15. The method according to any one of embodiments 1 to 14, wherein the subject's immune cells are maintained at levels corresponding with their immune cells prior to administration of the anti-KMA antibody.
16. The method according to any one of embodiments 11 to 14, wherein the subject's immune cells are maintained at levels corresponding with their immune cells after the treatment phase.
17. The method according to embodiment 15, wherein the subject's immune cells are one or more or all of red blood cells; white blood cells; and platelets.
18. The method according to any one of embodiments 11 to 17, further comprising a maintenance phase which comprises administering to the subject: (i) at least 30 mg/kg of the anti-KMA antibody once per month; (ii) the IMiD or the CELMOD once per day; and (iii) the steroid once per week, wherein the maintenance phase follows after the treatment phase.
19. The method according to any one of embodiments 11 to 17, further comprising a maintenance phase which comprises administering to the subject at least 30 mg/kg of the anti-KMA antibody once per month.
20. The method according to embodiment 18 or 19, wherein the duration of the maintenance phase is at least 12 weeks.
21. The method according to any one of embodiments 18-20, wherein the maintenance phase continues until disease progression.
22. The method according to any one of embodiments 1 to 21, wherein about 30 mg/kg of the anti-KMA antibody is administered to the subject.
23. The method according to any one of embodiments 1 to 22, wherein the anti-KMA antibody is formulated for intravenous infusion.
24. The method according to any one of embodiments 1 to 23, wherein the subject has received at least one, at least two, at least three, at least four, at least five or at least six prior lines of therapy.
25. The method according to any one of embodiments 1 to 24, wherein the subject achieved at least a minimal response (25% reduction in M protein) to their most recent line of therapy.
26. The method according to any one of embodiments 1 to 25, wherein the subject is refractory to at least one, at least two, at least three or at least four prior lines of therapy.
27. The method according to any one of embodiments 1 to 26, wherein the subject is refractory to at least one proteasome inhibitor, IMiD or CELMOD or autologous stem cell transplantation.
28. The method according to any one of embodiments 1 to 27, wherein the subject has
relapsed myeloma.
29. The method according to any one of embodiments 1 to 28, wherein the subject's multiple myeloma has relapsed and is refractory to at least one proteasome inhibitor, IMiD or CELMOD or autologous stem cell transplantation.
   30. The method according to any one of embodiments 1 to 12 and 14 to 28, wherein the subject has no prior exposure to the IMiD or the CELMOD.
   31. A combination dosing regimen for use in the treatment of multiple myeloma in a subject comprising (i) at least 30 mg/kg of an anti-KMA antibody; (ii) an immunomodulatory drug (IMiD) or cereblon E3 ligase modulator (CELMOD); and (iii) a steroid.
   32. The combination dosing regimen according to embodiment 31, wherein about 25 mg of the IMiD or the CELMOD is administered to the subject.
   33. The combination dosing regimen according to embodiment 31 or 32, wherein about 40 mg of the steroid is administered to the subject.
   34. The combination dosing regimen according to any one of embodiments 31 to 33, wherein the combination is administered once per week in a treatment phase.
   35. The combination dosing regimen according to any one of embodiments 31 to 34, wherein the treatment phase comprises administration of: (i) at least 30 mg/kg of the anti-KMA antibody once per week; (ii) the IMiD or the CELMOD once per day; and (iii) the steroid once per week.
   36. The combination dosing regimen according to any one of embodiments 31 to 35, wherein the duration of the treatment phase is between at least 6 weeks and at least 10 weeks.
   37. The combination dosing regimen according to any one of embodiments 31 to 36, further comprising administering to the subject the IMiD or the CELMOD once per day for at least 3 weeks prior to the start of the treatment phase.
   38. The combination dosing regimen according to any one of embodiments 31 to 37, further comprising administering a steroid to the subject at least 1 week prior to the start of the treatment phase.
   39. The combination dosing regimen according to any one of embodiments 31 to 37, further comprising administering an anti-KMA antibody to the subject once per week for at least 1 week, at least 2 weeks, at least 3 weeks, at least 4 weeks, at least 5 weeks, at least 6 weeks, at least 7 weeks or at least 8 weeks prior to the start of the treatment phase.
   40. The combination dosing regimen according to any one of embodiments 31 to 39, further comprising a maintenance phase which comprises administering to the subject: (i) at least 30 mg/kg of the anti-KMA antibody once per month; (ii) the IMiD or the CELMOD once per day; and (iii) the steroid once per week, wherein the maintenance phase follows after the treatment phase.
   41. The combination dosing regimen according to any one of embodiments 31 to 39, further comprising a maintenance phase which comprises administering to the subject at least 30 mg/kg of the anti-KMA antibody once per month.
   42. A kit comprising: (i) a single dose or multiple doses of at least 30 mg/kg of an anti-KMA antibody; (ii) a single dose or multiple doses of an immunomodulatory drug (IMiD) or cereblon E3 ligase modulator (CELMOD);
   (iii) a single dose or multiple doses of a steroid; (iv) instructions for using the anti-KMA antibody, the IMiD or the CELMOD, and the steroid, for the treatment of multiple myeloma in a subject.
   43. A method of adjuvant therapy comprising administering to a subject having multiple myeloma, following or in combination with a primary treatment, an effective amount of an anti-KMA antibody.
   44. The method according to embodiment 43, wherein the effective amount is about 30 mg/kg to about 60 mg/kg of the anti-KMA antibody.
   45. The method according to embodiment 43, wherein the effective amount is 30 mg/kg to 60 mg/kg of the anti-KMA antibody.
   46. The method according to embodiment 43, wherein the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody.
   47. The method according to embodiment 43, wherein the effective amount is at least 10 mg/kg of the anti-KMA antibody.
   48. The method according to embodiment 43, wherein the effective amount is at least 30 mg/kg of the anti-KMA antibody.
   49. The method according to embodiment 43, wherein the effective amount is at least 45 mg/kg of the anti-KMA antibody.
   50. The method according to embodiment 43, wherein the effective amount is at least 60 mg/kg of the anti-KMA antibody.
   51. The method according to any one of embodiments 43-50, wherein the primary treatment is at least one proteasome inhibitor, IMiD, CELMOD or autologous stem cell transplantation.
   52. The method according to any one of embodiments 43-50, wherein the primary treatment comprises a combination of an IMiD or a CELMOD and a steroid.
   53. The method according to embodiment 52, wherein about 25 mg of the IMiD or the CELMOD is administered to the subject.
   54. The method according to embodiment 52 or 53, wherein about 40 mg of the steroid is administered to the subject.
   55. The method according to any one of embodiments 43-54, wherein the IMiD is lenalidomide, pomalidomide or thalidomide.
   56. The method according to embodiment 55, wherein the IMiD is lenalidomide.
   57. The method according to embodiment 55, wherein the IMiD is pomalidomide.
   58. The method according to any one of embodiments 43-57, wherein the steroid is dexamethasone or prednisone.
   59. The method according to any one of embodiments 43-57, wherein the steroid is dexamethasone.
   60. The method according to any one of embodiments 43-59, wherein the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks.
   61. The method according to any one of embodiments 43-59, wherein the anti-KMA antibody is administered once per month for at least 1 month, at least 2 months, at least 3 months, at least 4 months, at least 5 months or at least 6 months.
   62. The method according to any one of embodiments 43-59, wherein the anti-KMA antibody is administered for 12 weeks.
   63. The method according to any one of embodiments 43-59, wherein the anti-KMA antibody is administered until disease progression.
   64. An adjuvant method of treating multiple myeloma in a subject, the method comprising administering an effective amount of an anti-KMA antibody in combination with an existing therapeutic protocol.
   65. The method according to embodiment 64, wherein the effective amount is about 30 mg/kg to about 60 mg/kg of the anti-KMA antibody.
   66. The method according to embodiment 64, wherein the effective amount is 30 mg/kg to 60 mg/kg of the anti-KMA antibody.
   67. The method according to embodiment 64, wherein the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody.
   68. The method according to embodiment 64, wherein the effective amount is at least 10 mg/kg of the anti-KMA antibody.
   69. The method according to embodiment 64, wherein the effective amount is at least 30 mg/kg of the anti-KMA antibody.
   70. The method according to embodiment 64, wherein the effective amount is at least 45 mg/kg of the anti-KMA antibody.
   71. The method according to embodiment 64, wherein the effective amount is at least 60 mg/kg of the anti-KMA antibody.
   72. The method according to any one of embodiments 64-71, wherein the existing therapeutic protocol comprises administering a combination of an IMiD or a CELMOD and a steroid.
   73. The method according to embodiment 72, wherein about 25 mg of the IMiD or the CELMOD is administered to the subject.
   74. The method according to embodiment 72 or 73, wherein about 40 mg of the steroid is administered to the subject.
   75. The method according to any one of embodiments 72-74, wherein the IMiD is lenalidomide, pomalidomide or thalidomide.
   76. The method according to embodiment 75, wherein the IMiD is pomalidomide.
   77. The method according to any one of embodiments 64-76, wherein the steroid is dexamethasone or prednisone.
   78. The method according to any one of embodiments 72-77, wherein the steroid is dexamethasone.
   79. The method according to any one of embodiments 64-78, wherein the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks.
   80. The method according to any one of embodiments 64-78, wherein the anti-KMA antibody is administered once per month for at least 1 month, at least 2 months, at least 3 months, at least 4 months, at least 5 months or at least 6 months.
   81. The method according to any one of embodiments 64-78, wherein the anti-KMA antibody is administered for 12 weeks.
   82. The method according to any one of embodiments 64-78, wherein the anti-KMA antibody is administered until disease progression.
   83. The method according to any one of embodiments 64-82, wherein the multiple myeloma (MM) is a relapsed or refractory MM.
   84. The method according to any one of embodiments 64-82, wherein the multiple myeloma is relapsed refractory multiple myeloma (RRMM).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

All publications, patents and patent applications herein are incorporated by reference to the same extent as if each individual publication or patent application was specifically and individually indicated to be incorporated by reference. It is not an admission that any of the information provided herein is prior art or relevant to the presently claimed inventions, or that any publication specifically or implicitly referenced is prior art.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

While the invention has been described in connection with specific embodiments thereof, the foregoing description has been given for clearness of understanding only and no unnecessary limitations should be understood therefrom. It will be understood that the description is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. Those skilled in the art will recognize or be able to ascertain using no more than routine experimentation, many equivalents to the specific embodiments of the invention described herein. Such equivalents are intended to be encompassed by the following claims.

1. A method of treating multiple myeloma in a subject in need thereof, the method comprising administering to the subject a treatment which comprises:
(i) at least 30 mg/kg of an anti-KMA antibody;
(ii) an immunomodulatory drug (IMiD) or cereblon E3 ligase modulator (CELMOD); and
(iii) a steroid.

2. The method according to claim 1, wherein the anti-KMA antibody comprises a VH and a VL, the VH comprising a complementarity determining region (CDR) 1 comprising an amino acid sequence as shown in SEQ ID NO: 6, a CDR2 comprising an amino acid sequence as shown in SEQ ID NO: 7 and a CDR3 comprising a sequence as shown in SEQ ID NO: 8 and the VL comprising a CDR 1 comprising an amino acid sequence as shown in SEQ ID NO: 9, a CDR2 comprising an amino acid sequence as shown in SEQ ID NO: 10 and a CDR3 comprising a sequence as shown in SEQ ID NO: 11.

3. The method according to claim 1 or 2, wherein the anti-KMA antibody has a heavy chain variable region (VH) comprising a sequence set forth in SEQ ID NO: 1 and a light chain variable region (VL) comprising a sequence set forth in SEQ ID NO: 2.

4. The method according to any one of claims 1 to 3, wherein the IMiD is lenalidomide, pomalidomide or thalidomide.

5. The method according to any one of claims 1 to 4, wherein the IMiD is pomalidomide.

6. The method according to any one of claims 1 to 3, wherein the CELMOD is iberdomide or CC-92480.

7. The method according to any one of claims 1 to 6, wherein about 25 mg of the IMiD or the CELMOD is administered to the subject.

8. The method according to any one of claims 1 to 7, wherein the steroid is dexamethasone or prednisone.

9. The method according to any one of claims 1 to 8, wherein the steroid is dexamethasone.

10. The method according to any one of claims 1 to 9, wherein about 40 mg of the steroid is administered to the subject.

11. The method according to any one of claims 1 to 10, wherein treatment comprises administering to the subject in a treatment phase:
(i) at least 30 mg/kg of the anti-KMA antibody once per week;
(ii) the IMiD or the CELMOD once per day; and
(iii) the steroid once per week.

12. The method according to claim 11, wherein the duration of the treatment phase is between at least 6 weeks and at least 10 weeks.

13. The method according to claim 11 or 12, further comprising administering to the subject the IMiD or the CELMOD once per day for at least 3 weeks prior to the start of the treatment phase.

14. The method according to any one of claims 11 to 13, further comprising administering a steroid to the subject at least 1 week prior to the start of the treatment phase.

15. The method according to any one of claims 1 to 14, wherein the subject's immune cells are maintained at levels corresponding with their immune cells prior to administration of the anti-KMA antibody.

16. The method according to any one of claims 11 to 14, wherein the subject's immune cells are maintained at levels corresponding with their immune cells after the treatment phase.

17. The method according to claim 15, wherein the subject's immune cells are one or more or all of red blood cells; white blood cells; and platelets.

18. The method according to any one of claims 11 to 17, further comprising a maintenance phase which comprises administering to the subject:
(i) at least 30 mg/kg of the anti-KMA antibody once per month;
(ii) the IMiD or the CELMOD once per day; and
(iii) the steroid once per week,
wherein the maintenance phase follows after the treatment phase.

19. The method according to any one of claims 11 to 17, further comprising a maintenance phase which comprises administering to the subject at least 30 mg/kg of the anti-KMA antibody once per month.

20. The method according to claim 18 or 19, wherein the duration of the maintenance phase is at least 12 weeks.

21. The method according to any one of claims 18-20, wherein the maintenance phase continues until disease progression.

22. The method according to any one of claims 1 to 21, wherein about 30 mg/kg of the anti-KMA antibody is administered to the subject.

23. The method according to any one of claims 1 to 22, wherein the anti-KMA antibody is formulated for intravenous infusion.

24. The method according to any one of claims 1 to 23, wherein the subject has received at least one, at least two, at least three, at least four, at least five or at least six prior lines of therapy.

25. The method according to any one of claims 1 to 24, wherein the subject achieved at least a minimal response (25% reduction in M protein) to their most recent line of therapy.

26. The method according to any one of claims 1 to 25, wherein the subject is refractory to at least one, at least two, at least three or at least four prior lines of therapy.

27. The method according to any one of claims 1 to 26, wherein the subject is refractory to at least one proteasome inhibitor, IMiD or CELMOD or autologous stem cell transplantation.

28. The method according to any one of claims 1 to 27, wherein the subject has relapsed myeloma.

29. The method according to any one of claims 1 to 28, wherein the subject's multiple myeloma has relapsed and is refractory to at least one proteasome inhibitor, IMiD or CELMOD or autologous stem cell transplantation.

30. The method according to any one of claims 1 to 12 and 14 to 28, wherein the subject has no prior exposure to the IMiD or the CELMOD.

31. A combination dosing regimen for use in the treatment of multiple myeloma in a subject comprising:
(i) at least 30 mg/kg of an anti-KMA antibody;
(ii) an immunomodulatory drug (IMiD) or cereblon E3 ligase modulator (CELMOD); and
(iii) a steroid.

32. The combination dosing regimen according to claim 31, wherein about 25 mg of the IMiD or the CELMOD is administered to the subject.

33. The combination dosing regimen according to claim 31 or 32, wherein about 40 mg of the steroid is administered to the subject.

34. The combination dosing regimen according to any one of claims 31 to 33, wherein the combination is administered once per week in a treatment phase.

35. The combination dosing regimen according to any one of claims 31 to 34, wherein the treatment phase comprises administration of:
(i) at least 30 mg/kg of the anti-KMA antibody once per week;
(ii) the IMiD or the CELMOD once per day; and
(iii) the steroid once per week.

36. The combination dosing regimen according to any one of claims 31 to 35, wherein the duration of the treatment phase is between at least 6 weeks and at least 10 weeks.

37. The combination dosing regimen according to any one of claims 31 to 36, further comprising administering to the subject the IMiD or the CELMOD once per day for at least 3 weeks prior to the start of the treatment phase.

38. The combination dosing regimen according to any one of claims 31 to 37, further comprising administering a steroid to the subject at least 1 week prior to the start of the treatment phase.

39. The combination dosing regimen according to any one of claims 31 to 37, further comprising administering an anti-KMA antibody to the subject once per week for at least 1 week, at least 2 weeks, at least 3 weeks, at least 4 weeks, at least 5 weeks, at least 6 weeks, at least 7 weeks or at least 8 weeks prior to the start of the treatment phase.

40. The combination dosing regimen according to any one of claims 31 to 39, further comprising a maintenance phase which comprises administering to the subject:
(i) at least 30 mg/kg of the anti-KMA antibody once per month;
(ii) the IMiD or the CELMOD once per day; and
(iii) the steroid once per week,
wherein the maintenance phase follows after the treatment phase.

41. The combination dosing regimen according to any one of claims 31 to 39, further comprising a maintenance phase which comprises administering to the subject at least 30 mg/kg of the anti-KMA antibody once per month.

42. A kit comprising:
(i) a single dose or multiple doses of at least 30 mg/kg of an anti-KMA antibody;
(ii) a single dose or multiple doses of an immunomodulatory drug (IMiD) or cereblon E3 ligase modulator (CELMOD);
(iii) a single dose or multiple doses of a steroid;
(iv) instructions for using the anti-KMA antibody, the IMiD or the CELMOD, and the steroid, for the treatment of multiple myeloma in a subject.

43. A method of adjuvant therapy comprising administering to a subject having multiple myeloma, following or in combination with a primary treatment, an effective amount of an anti-KMA antibody.

44. The method according to claim 43, wherein the effective amount is about 30 mg/kg to about 60 mg/kg of the anti-KMA antibody.

45. The method according to claim 43, wherein the effective amount is 30 mg/kg to 60 mg/kg of the anti-KMA antibody.

46. The method according to claim 43, wherein the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody.

47. The method according to claim 43, wherein the effective amount is at least 10 mg/kg of the anti-KMA antibody.

48. The method according to claim 43, wherein the effective amount is at least 30 mg/kg of the anti-KMA antibody.

49. The method according to claim 43, wherein the effective amount is at least 45 mg/kg of the anti-KMA antibody.

50. The method according to claim 43, wherein the effective amount is at least 60 mg/kg of the anti-KMA antibody.

51. The method according to any one of claims 43-50, wherein the primary treatment is at least one proteasome inhibitor, IMiD, CELMOD or autologous stem cell transplantation.

52. The method according to any one of claims 43-50, wherein the primary treatment comprises a combination of an IMiD or a CELMOD and a steroid.

53. The method according to claim 52, wherein about 25 mg of the IMiD or the CELMOD is administered to the subject.

54. The method according to claim 52 or 53, wherein about 40 mg of the steroid is administered to the subject.

55. The method according to any one of claims 43-54, wherein the IMiD is lenalidomide, pomalidomide or thalidomide.

56. The method according to claim 55, wherein the IMiD is lenalidomide.

57. The method according to claim 55, wherein the IMiD is pomalidomide.

58. The method according to any one of claims 43-57, wherein the steroid is dexamethasone or prednisone.

59. The method according to any one of claims 43-57, wherein the steroid is dexamethasone.

60. The method according to any one of claims 43-59, wherein the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks.

61. The method according to any one of claims 43-59, wherein the anti-KMA antibody is administered once per month for at least 1 month, at least 2 months, at least 3 months, at least 4 months, at least 5 months or at least 6 months.

62. The method according to any one of claims 43-59, wherein the anti-KMA antibody is administered for 12 weeks.

63. The method according to any one of claims 43-59, wherein the anti-KMA antibody is administered until disease progression.

64. An adjuvant method of treating multiple myeloma in a subject, the method comprising administering an effective amount of an anti-KMA antibody in combination with an existing therapeutic protocol.

65. The method according to claim 64, wherein the effective amount is about 30 mg/kg to about 60 mg/kg of the anti-KMA antibody.

66. The method according to claim 64, wherein the effective amount is 30 mg/kg to 60 mg/kg of the anti-KMA antibody.

67. The method according to claim 64, wherein the effective amount is at least 0.03 mg/kg, at least 1 mg/kg, at least 3 mg/kg, at least 10 mg/kg, at least 30 mg/ml, at least 45 mg/ml or at least 60 mg/kg of the anti-KMA antibody.

68. The method according to claim 64, wherein the effective amount is at least 10 mg/kg of the anti-KMA antibody.

69. The method according to claim 64, wherein the effective amount is at least 30 mg/kg of the anti-KMA antibody.

70. The method according to claim 64, wherein the effective amount is at least 45 mg/kg of the anti-KMA antibody.

71. The method according to claim 64, wherein the effective amount is at least 60 mg/kg of the anti-KMA antibody.

72. The method according to any one of claims 64-71, wherein the existing therapeutic protocol comprises administering a combination of an IMiD or a CELMOD and a steroid.

73. The method according to claim 72, wherein about 25 mg of the IMiD or the CELMOD is administered to the subject.

74. The method according to claim 72 or 73, wherein about 40 mg of the steroid is administered to the subject.

75. The method according to any one of claims 72-74, wherein the IMiD is lenalidomide, pomalidomide or thalidomide.

76. The method according to claim 75, wherein the IMiD is pomalidomide.

77. The method according to any one of claims 64-76, wherein the steroid is dexamethasone or prednisone.

78. The method according to any one of claims 72-77, wherein the steroid is dexamethasone.

79. The method according to any one of claims 64-78, wherein the anti-KMA antibody is administered once per week for at least 4 weeks, at least 8 weeks, at least 12 weeks, at least 16 weeks, at least 20 weeks, at least 24 weeks, at least 28 weeks, at least 32 weeks or at least 36 weeks.

80. The method according to any one of claims 64-78, wherein the anti-KMA antibody is administered once per month for at least 1 month, at least 2 months, at least 3 months, at least 4 months, at least 5 months or at least 6 months.

81. The method according to any one of claims 64-78, wherein the anti-KMA antibody is administered for 12 weeks.

82. The method according to any one of claims 64-78, wherein the anti-KMA antibody is administered until disease progression.

83. The method according to any one of claims 64-82, wherein the multiple myeloma (MM) is a relapsed or refractory MM.

84. The method according to any one of claims 64-82, wherein the multiple myeloma is relapsed refractory multiple myeloma (RRMM).


SEQUENCE LISTING (13 sequences)

SEQ ID NO: 1 - protein, 449 aa, synthetic construct
EVQLQQSGAELVKPGASVKLSCTASGFNIKDTYMHWVKQRPEQGLEWIGRIDPANGNTKYDPKFQGKATIIADTSSNTAYLQLSSLTSEDTAVYYCARGVYHDYDGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 111 aa, synthetic construct
LEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, synthetic construct
LDIKR

SEQ ID NO: 5 - protein, 5 aa, synthetic construct
LEIKR

SEQ ID NO: 6 - protein, 11 aa, synthetic construct
GFNIKDTYMHW

SEQ ID NO: 7 - protein, 17 aa, synthetic construct
RIDPANGNTKYDPKFQG

SEQ ID NO: 8 - protein, 10 aa, synthetic construct
GVYHDYDGDY

SEQ ID NO: 9 - protein, 11 aa, synthetic construct
KASQNVGTNVA

SEQ ID NO: 10 - protein, 7 aa, synthetic construct
STSYRYS

SEQ ID NO: 11 - protein, 9 aa, synthetic construct
QQYNSYPYT

SEQ ID NO: 12 - protein, 119 aa, synthetic construct
QVQLQQSGAELVKPGASVKLSCTASGFNIKDTYMHWVKQRPEQGLEWIGRIDPANGNTKYDPKFQGKAAIIADTSSNTAYLQLSSLTSEDTAVYYCARGVYHDYDGDYWGQGTTVTVAS

SEQ ID NO: 13 - protein, 107 aa, synthetic construct
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIK